(19) United States
(12) Patent Application Publication
(10) Pub. No.: US 2026/0224780 A1
(43) Pub. Date: Aug. 6, 2026

(54) BIOMATERIAL COMPRISING AT LEAST ONE ELASTOMERIC MATRIX AND A NON-SULFATED POLYSACCHARIDE AND USES THEREOF

(71) Applicants: UNIVERSITE PARIS CITE, PARIS (FR); UNIV. PARIS XIII PARIS-NORD VILLETANEUSE, VILLETANEUSE (FR); CENTRE NATIONAL DE LA RECHERCHE SCIENTIFIQUE, Paris (FR); UNIVERSITE ST-JOSEPH DE BEYROUTH, BEYROUTH (LB)

(72) Inventors: Didier LUTOMSKI, MONTIGNY LE BRETONNEUX (FR); Geraldine ROHMAN, DEUIL LA BARRE (FR); Sylvie CHANGOTADE, OTHIS (FR); Karim SENNI, AULNAY SOUS BOIS (FR); Carole CHAKAR, AOUKAR, AL HADIRA, EL METN (LB); Wassim MANHAL, JDEIDEH EL MATEN, BEYROUTH (LB); Ronald YOUNES, BEYROUTH (LB)

(21) Appl. No.: 19/573,505

(22) Filed: Mar. 20, 2026

Related U.S. Application Data
(62) Division of application No. 17/997,433, filed on May 20, 2021, now abandoned.

(30) Foreign Application Priority Data
May 20, 2020 (FR) 2005382

(51) Int. Cl. A61L 27/26; A61L 27/54; A61L 27/56

(52) U.S. Cl. CPC A61L 27/26; A61L 27/54; A61L 27/56; A61L 2300/236; A61L 2430/34

(57) ABSTRACT

The present invention relates to a biomaterial comprising at least one elastomeric matrix and a polysaccharide, and to uses thereof in the reinforcement, reconstruction and/or filling in of tissue defects, preferably the reinforcement, reconstruction and/or filling in of soft tissue defects and/or epithelial tissues, preferably skin and/or mucosal repair.

### CROSS-REFERENCE TO RELATED APPLICATIONS

This application is a divisional of U.S. application Ser. No. 17/997,433, filed Oct. 28, 2022, which is a U.S. Natl. Stage of International Appln. PCT/EP2021/063472, filed May 20, 2021, which claims the benefit of French Appln. 2005382, filed May 20, 2020, all of which are hereby incorporated by reference.

### TECHNICAL FIELD

The present invention relates to a biomaterial and its uses in tissue reinforcement, reconstruction and/or filling, preferably reinforcement, reconstruction and/or filling of soft tissue defects and/or epithelial tissues, preferably repair of skin, gum and/or mucosa.

### PRIOR ART

“Mucogingival” periodontal surgery answers problems associated with deficits in soft gingiva-mucosal tissues inducing functional, aesthetic and biological incidence both around natural teeth and dental implants.

For example, gingival recession results in an unaesthetic aspect of the smile, it may also be the source of spontaneous and/or provoked hypersensitivity, promote the development of deep caries and cause a functional discomfort linked to the presence of periodontal inflammation that can endanger the tooth supporting tissues (periodontium) allowing the anchoring thereof in the maxillary and mandible bony bases.

This periodontal disease results in movement of the marginal gum and the epithelio-conjunctival attachment junction apically relative to the cementoenamel junction.

Their etiology is multifactorial and linked to several predisposition factors (fine biotype, bone dehiscences, low height and thickness of keratinized tissue, dental malposition, etc.), mechanical factors such as traumatic toothbrushing, bacteriological factors (presence of plaque and inflammation) or other factors such as occlusal trauma, smoking and the like).

Soft tissue defects, particularly oral and dental soft tissues, may result from trauma or surgical ablations frequently causing loss of the original anatomical structure of the soft tissue.

In addition, changes in soft tissues negatively affect the patient's aesthetic appearance and therefore their satisfaction.

Depending on the size of the defect, tissue deformations can be aesthetically corrected by soft tissue augmentation or by soft tissue reconstruction or by surgical techniques.

This tissue management can also incorporate other indications such as peri-implant tissues and the arrangements of maxilla crests.

To resolve this tissue deficiency, a surgical tissue graft technique must be considered. Multiple surgical approaches documented in the literature have been proposed to achieve root coverage (or exposed implant surfaces coverage) for gum recession treatment, tissue thickening so as to obtain a thick biotype and augmentation of keratinized gum band, necessary to long-term periodontal and peri-implant environment durability. Most of these techniques require a second palatal surgical site, (autograft taken from the mouth, which extends the intervention time and can be the source of harmful postoperative consequences, with many drawbacks (pain, abundant bleeding during or after surgery, morbidity, delayed healing, bone necrosis, paresthesia or palatal permanent anesthesia have been reported). These drawbacks are sometimes associated with anatomical limitations in case of too fine palate, unsuitable for providing a sufficient amount of tissue, or with medical care refusal when the patient opposes a tissue sample taken from another “donor” site which is painful and can be responsible for complications. The first solution considered for repairing soft tissue defects is the grafting of a connective tissue fraction taken elsewhere in the patient's body.

This is then referred to as connective tissue autograft. The autograft produces no immune response, since the tissue comes from the patient. However, it results in considerable cell death in the transplanted tissue. The ability of the graft to produce new cells can compensate for this loss, but it depends in particular on the graft vascularization. The latter is in fact essential to the tissue reconstruction: the blood vessels supply the energy and the nutrients necessary for cell proliferation. Furthermore, autografting requires two surgical sites (harvesting and then grafting) which can cause complications (pain, abscesses, neuralgia). The size of the graft required for filling represents another important limit.

Another alternative is to use an allogeneic substitute.

An allodermal substitute conventionally used by practitioners for reconstructing soft tissues and/or filling in the defects of soft tissues is the AlloDerm® product marketed by the Biohorizon company. AlloDerm® is a cell-free dermal matrix of human origin, obtained from the human donors cadavers skin, having undergone a physical and chemical treatment involving a tissue deepidermization, which induces the separation of the anchoring fibrils of the hemidesmosomes from the basal keratinocytes by eliminating all the cellular content (epithelial, connective, viral and bacterial cells), which implies the removal of the epidermal layer with all the cellular components without damaging the components of the connective tissue matrix, under conditions which do not alter the collagen fibers beams or damage the basement membrane complex. This process leaves behind it extracellular collagen which provides the basis for cell growth followed by tissue remodelling.

Another allogeneic substitute conventionally used by practitioners for soft tissue reconstruction and/or filling in soft tissue defects is the Mucoderm® product, marketed by Botiss.

Mucoderm® is a matrix based on natural collagen of type I/III derived from porcine dermis and elastin.

However, these products have many disadvantages. Indeed, these products have a relatively high cost and require very long postoperative monitoring and early exposure of the matrix can limit the graft vascularization, consequently resulting in a reduction in the potential for covering the recessions. In addition, Mucoderm® undergoes a necrotic process. The healing and the substitution of AlloDerm® or Mucoderm® by a newly formed tissue are particularly slow, of the order of 10 weeks. In fact, because of its non-vital structure, the healing and the substitution of AlloDerm® or Mucoderm® depend on the cells and blood vessels present in the surrounding tissues, which leads to slowing down the incorporation, which may result in structural and functional irregularities. In addition, their macromolecular structure is different from the one of a physiological gum, despite an assumed similar macromolecular composition. Indeed, the highly dense collagen network of the allogeneic substitute seems to limit cellular colonization in vitro and tissue remodelling in vitro and in vivo. In fact, the gingival extracellular matrix is constantly remodeled, in order to resist mechanical stresses. However, the observed fibrotic process is due to the non-physiological remodeling of the gum at the graft site. In addition, the continuous presence of foreign multinucleated giant cells could induce poor integration of the allogeneic substitute and persistent clinical redness. In addition, it has been demonstrated that Mucoderm® once grafted shrinks during healing during which a remodeling process takes place which can result in contraction of the wound.

In addition, it has been demonstrated that Mucoderm® presents crumbling issues when the implant is subjected to high mechanical stresses and causes high inflammatory responses. Last, the animal origin of certain allogeneic substitutes may sometimes result in a rejection for religious or philosophical convictions.

Other fields of surgery are also in search of biocompatible materials allowing to fill tissue defects or losses, due to trauma (burn, (peel, tear), ageing or pathologies; or allowing to reinforce tissues following trauma, ageing or a pathology. For example, many companies are specialized in the design of implants used as reinforcement in gynaecological, urinary or visceral (or parietal) surgery. These materials may be designed for the treatment of vascular wounds, digestive wounds, eventrations, etc. These biomaterials can thus be used for the design of reinforcing implants for treatment of pelvic organs prolapse and more particularly in the treatment of pelvic organs prolapse in women (anterior (urinary, cystocele, stress incontinence), medium (genital, colpocele) and/or posterior (digestive rectocele)), or Peyronie's disease in human beings. The biomaterial can then be declined in the form of an extensible reinforcing sheet, membrane or implant wick of any shape. The biomaterials used with more or less success are currently of xenogeneic origin (such as Pelvicol®, marketed by Bard France SAS) or synthetic origin (polypropylene such as Parietex®, marketed by SOFRADIM). However, Pelvicol® presents crumbling problems when the implant is subjected to high mechanical stresses and causes high inflammatory responses.

Document US2012/239161 describes an elastomer matrix based on caprolactone and agar or gelatin.

Document CN 108034225 describes a process for preparing a composite material comprising an elastomeric matrix and chitosan.

This is the reason why there is a need to provide a new biomaterial able to reinforce, reconstruct and/or fill in tissue defects, easy to use for practitioners, and which has mechanical properties suitable for implantation in soft tissues in terms of elasticity and volume conservation. There is also a need for a biomaterial having good biocompatibility and a degradability suitable for tissue regeneration. It is also a matter of providing a biomaterial which is not of animal origin.

### SUMMARY OF THE INVENTION

Thus, the subject of the present invention is therefore a biomaterial for tissue repair comprising:
at least one elastomer matrix, and
a non-sulfated saccharide polymer.

Another subject of the present invention is the use of said biomaterial in tissue repair, preferably soft tissue and/or epithelial tissue repair, preferably skin and/or mucosa repair.

Another subject of the present invention is a method for preparing a biomaterial.

### DETAILED DESCRIPTION

Thus, the subject of the present invention is a biomaterial for tissue repair comprising:
at least one elastomer matrix, and
a non-sulfated saccharide polymer.

The invention has the advantage of providing a porous bioresorbable/biodegradable elastomeric biomaterial promoting cell migration and vascularization. The biomaterial according to the invention also offers better tissue biointegration without any risk of microbial contamination.

Within the meaning of the present invention, the term “biomaterial” is intended to mean a material used and suitable for a medical application. Advantageously, the biomaterial according to the invention is a physical support on which fibroblasts can adhere, migrate and proliferate at the surface and inside said physical support, said physical support being capable of resorbing or of being biodegradable, thus allowing its substitution by the newly formed connective tissue.

Advantageously, the biomaterial according to the invention comprises at least an elastomer matrix and a non-sulfated polysaccharide, the individual properties of which combine and having greatly improved overall performance, properties which cannot be observed with the at least one elastomer matrix or the non-sulfated polysaccharide, used individually.

The inventors have surprisingly shown that the biomaterial comprising at least an elastomer matrix and a non-sulfated polysaccharide according to the invention has:
mechanical properties sufficient to withstand the force stresses exerted by the cells, but also the regeneration process in the zone to be repaired and to be a support for the soft tissue in this zone,
a porosity and an interconnectivity allowing circulation of fibroblasts, nutrients and other molecules involved in the regularization of these processes, while allowing internal vascularization of the biomaterial of the invention,
a roughness allowing cell adhesion and adsorption of molecules involved in the regularization of these processes.

Advantageously, the inventors have shown that the biomaterial when implanted in the patient can activate collagen synthesis and vascularization, allowing rapid reconstruction of the damaged tissue.

In a particular embodiment of the invention, the non-sulfated polysaccharide may be attached by a covalent bond to the elastomer matrix. In another particular embodiment of the invention, the non-sulfated polysaccharide can be dispersed in and at the surface of the elastomer matrix.

Within the meaning of the present invention, the term “elastomeric matrix” is intended to mean a structure consisting of a single elastomer or of a combination of two or more elastomer systems, said structure being capable of including the non-sulfated polysaccharide.

Advantageously, the isocyanate index of the elastomer matrix is between 0.1 and 6.0.

Advantageously, the isocyanate index is between 0.1 and 5.0, advantageously between 0.2 and 4.9, advantageously between 0.3 and 4.8, advantageously between 0.4 and 4.7, advantageously between 0.5 and 4.7, advantageously between 0.6 and 4.6, advantageously between 0.7 and 4.5, advantageously between 0.8 and 4.5, advantageously between 0.9 and 4.5, advantageously between 1 and 4.5, advantageously between 1.05 and 4.5, advantageously between 1.1 and 4.5, advantageously between 1.2 and 4.5, advantageously between 1.3 and 4.5, advantageously between 1.4 and 4.5, advantageously between 1.5 and 4.5, advantageously between 2.0 and 4.5, advantageously between 2.5 and 4.5, advantageously between 2.6 and 4.4, advantageously between 2.7 and 4.3, advantageously between 2.8 and 4.2, advantageously between 2.9 and 4.2, advantageously between 2.9 and 4.2, advantageously between 3.0 and 4.0.

Advantageously, the at least one elastomer matrix according to the present invention has a good biodegradability, a good biocompatibility and good mechanical properties.

Within the meaning of the present invention, the term “elastomer” means one or more polymers having “rubber elasticity” properties, obtained after crosslinking.

In a particular embodiment of the invention, the elastomer must be biocompatible and biodegradable.

Advantageously, the compression Young's modulus of the biomaterial of the invention is between 1 kPa and 1000 kPa, advantageously between 50 kPa and 900 kPa, advantageously between 50 kPa and 800 kPa, advantageously between 50 kPa and 700 kPa, advantageously between 50 kPa and 600 kPa, advantageously between 50 kPa and 500 kPa, advantageously between 100 kPa and 400 kPa.

Within the meaning of the present invention, the term “biocompatible” elastomer matrix means an elastomer matrix which is both compatible for implantation in a patient, and compatible for inclusion of the non-sulfated polysaccharide therein, and that is suitable for soft tissue reconstruction once the biomaterial is implanted in a human or animal patient.

Within the meaning of the present invention, the term “compatible for implantation in a patient” is intended to mean an elastomer matrix which has an advantageous benefit/risk ratio from the therapeutical point of view once implanted, for example within the meaning of Directive 2001/83/EC.

Within the meaning of the present invention, “compatible for inclusion of the non-sulfated polysaccharide” means an elastomer matrix which allows the incorporation of the non-sulfated polysaccharide, without or with little degradation of the activity of said non-sulfated polysaccharide in the elastomer matrix. Advantageously, the non-sulfated polysaccharide is incorporated into the elastomer matrix. In other words, the non-sulfated polysaccharide is directly integrated into the elastomer matrix during the manufacture of the biomaterial according to the invention.

Within the meaning of the present invention, the term “biodegradable” elastomer matrix is intended to mean an elastomer matrix which is bioresorbable and/or biodegradable and/or bioabsorbable, with a common goal of progressive disappearance, with one or more different or complementary mechanisms of degradation, solubilization or absorption of the elastomer matrix in the human or animal patient in which the material has been implanted.

In a particular embodiment of the invention, the at least one elastomer matrix according to the invention comprises a poly(ester-urea-urethane) based elastomer.

In a particularly advantageous embodiment of the invention, the at least one elastomer matrix of the biomaterial according to the invention comprises a poly(ester-urea-urethane) based elastomer, the ester being chosen from caprolactone oligomers (PCL), lactic acid oligomers (PLA), glycolic acid oligomers (PGA), hydroxybutyrate oligomers (PHB), hydroxyvalerate oligomers (PVB), dioxanone oligomers (PDO), poly(ethylene adipate) oligomers (PEA), poly(butylene adipate) oligomers (PBA) or combinations thereof.

In a particular embodiment, the at least one elastomer matrix of the porous biomaterial is a matrix comprising a poly(caprolactone-urea-urethane)-based elastomer.

In another particular embodiment, the at least one elastomer matrix of the porous biomaterial is a matrix comprising a poly(lactic acid-urea-urethane)-based elastomer.

In another particular embodiment, the at least one elastomer matrix of the porous biomaterial is a matrix comprising a poly(glycolic acid-urea-urethane)-based elastomer.

In another particular embodiment, the at least one elastomer matrix of the porous biomaterial is a matrix comprising a poly(hydroxyvalerate-urea-urethane)-based elastomer.

In another particular embodiment, the at least one elastomer matrix of the porous biomaterial is a matrix comprising a poly(hydroxybutyrate-urea-urethane)-based elastomer.

In another particular embodiment, the at least one elastomer matrix of the porous biomaterial is a matrix comprising a poly(dioxanone-urea-urethane)-based elastomer.

In another particular embodiment, the at least one elastomer matrix of the porous biomaterial is a matrix comprising a poly(ethylene adipate-urea-urethane)-based elastomer.

In another particular embodiment, the at least one elastomer matrix of the porous biomaterial is a matrix comprising a poly(butylene adipate urea urethane)-based elastomer.

In a particular embodiment, the at least one elastomer matrix of the porous biomaterial is a matrix comprising a poly(caprolactone-urea-urethane)-and poly(lactic acid-urea-urethane)-based elastomer.

In another particular embodiment, the at least one elastomer matrix of the porous biomaterial is a matrix comprising a poly(caprolactone-urea-urethane) and poly(glycolic acid-urea-urethane)-based elastomer.

In another particular embodiment, the at least one elastomer matrix of the porous biomaterial is a matrix comprising an elastomer based on poly(caprolactone-urea-urethane) and poly(hydroxyvalerate-urea-urethane).

In another particular embodiment, the at least one elastomer matrix of the porous biomaterial is a matrix comprising a poly(caprolactone-urea-urethane)-and poly(hydroxybutyrate-urea-urethane)-based elastomer. In another particular embodiment, the at least one elastomer matrix of the porous biomaterial is a matrix comprising an elastomer based on poly(caprolactone-urea-urethane) and poly(dioxanone-urea-urethane). In another particular embodiment, the at least one elastomer matrix of the porous biomaterial is a matrix comprising an elastomer based on poly(caprolactone-urea-urethane) and poly(ethylene adipate-urea-urethane). In another particular embodiment, the at least one elastomer matrix of the porous biomaterial is a matrix comprising an elastomer based on poly(caprolactone-urea-urethane) and poly(butylene adipate-urea-urethane).

In another particular embodiment, the at least one elastomer matrix of the porous biomaterial is a matrix comprising a poly(acid lactic-urea-urethane)-and poly(glycolic acid urea urethane)-based elastomer.

In another particular embodiment, the at least one elastomer matrix of the porous biomaterial is a matrix comprising a poly(lactic acid-urea-urethane)-and poly(hydroxyvalerate-urea-urethane)-based elastomer. In another particular embodiment, the at least one elastomer matrix of the porous biomaterial is a matrix comprising an elastomer based on poly(lactic acid-urea-urethane) and poly(hydroxybutyrate-urea-urethane).

In another particular embodiment, the at least one elastomer matrix of the porous biomaterial is a matrix comprising an elastomer based on poly(lactic acid-urea-urethane) and poly(dioxanone-urea-urethane). In another particular embodiment, the at least one elastomer matrix of the porous biomaterial is a matrix comprising an elastomer based on poly(lactic-urea-urethane acid) and poly(ethylene adipate-urea-urethane). In another particular embodiment, the at least one elastomer matrix of the porous biomaterial is a matrix comprising an elastomer based on poly(lactic-urea-urethane acid) and poly(butylene adipate-urea-urethane).

In another particular embodiment, the at least one elastomer matrix of the porous biomaterial is a matrix comprising an elastomer based on poly(glycolic acid-urea-urethane) and poly(hydroxyvalerate-urea-urethane).

In another particular embodiment, the at least one elastomer matrix of the porous biomaterial is a matrix comprising an elastomer based on poly(glycolic acid-urea-urethane) and poly(hydroxybutyrate-urea-urethane).

In another particular embodiment, the at least one elastomer matrix of the porous biomaterial is a matrix comprising an elastomer based on poly(glycolic acid-urea-urethane) and poly(dioxanone-urea-urethane).

In another particular embodiment, the at least one elastomer matrix of the porous biomaterial is a matrix comprising an elastomer based on poly(glycolic acid-urea-urethane) and poly(ethylene adipate-urea-urethane).

In another particular embodiment, the at least one elastomer matrix of the porous biomaterial is a matrix comprising an elastomer based on poly(glycolic acid-urea-urethane) and poly(butylene adipate-urea-urethane).

In another particular embodiment, the at least one elastomer matrix of the porous biomaterial is a matrix comprising an elastomer based on poly(hydroxyvalerate-urea-urethane) and poly(hydroxybutyrate-urea-urethane).

In another particular embodiment, the at least one elastomer matrix of the porous biomaterial is a matrix comprising an elastomer based on poly(hydroxyvalerate-urea-urethane) and poly(dioxanone-urea-urethane).

In another particular embodiment, the at least one elastomeric matrix of the porous biomaterial is an elastomeric matrix comprising an elastomer based on poly(hydroxyvalerate-urea-urethane) and poly(ethylene adipate-urea-urethane). In another particular embodiment, the at least one elastomer matrix of the porous biomaterial is a matrix comprising an elastomer based on poly(hydroxyvalerate-urea-urethane) and poly(butylene-adipate-urea urethane).

In another particular embodiment, the at least one elastomer matrix of the porous biomaterial is a matrix comprising an elastomer based on poly(hydroxybutyrate-urea-urethane) and poly(dioxanone-urea-urethane).

In another particular embodiment, the at least one elastomer matrix of the porous biomaterial is a matrix comprising an elastomer based on poly(hydroxybutyrate-urea-urethane) and poly(ethylene adipate-urea-urethane). In another particular embodiment, the at least one elastomer matrix of the porous biomaterial is a matrix comprising an elastomer based on poly(hydroxybutyrate-urea-urethane) and poly(butylene adipate-urea-urethane).

In another particular embodiment, the at least one elastomer matrix of the porous biomaterial is a matrix comprising an elastomer based on poly(dioxanone-urea-urethane) and poly(ethylene adipate-urea-urethane).

In another particular embodiment, the at least one elastomer matrix of the porous biomaterial is a matrix comprising an elastomer based on poly(dioxanone-urea-urethane) and poly(butylene adipate-urea-urethane).

In another particular embodiment, the at least one elastomer matrix of the porous biomaterial is a matrix comprising an elastomer based on poly(ethylene adipate-urea-urethane) and poly(butylene adipate-urea-urethane).

In another particular embodiment, the at least one elastomer matrix of the porous biomaterial is a matrix comprising an elastomer based on poly(caprolactone-urea-urethane), poly(lactic acid-urea-urethane) and poly(glycolic acid-urea-urethane).

In another particular embodiment, the at least one elastomer matrix of the porous biomaterial is a matrix comprising an elastomer based on poly(caprolactone-urea-urethane), poly(lactic acid-urea-urethane), poly(glycolic acid-urea-urethane) and poly(hydroxyvalerate-urea-urethane).

In another particular embodiment, the at least one elastomer matrix of the porous biomaterial is a matrix comprising an elastomer based on poly(caprolactone-urea-urethane), poly(lactic acid-urea-urethane), poly(glycolic acid-urea-urethane) and poly(hydroxybutyrate-urea-urethane).

In another particular embodiment, the at least one elastomer matrix of the porous biomaterial is a matrix comprising an elastomer based on poly(caprolactone-urea-urethane), poly(lactic acid-urea-urethane), poly(glycolic acid-urea-urethane) and poly(hydroxybutyrate-urea-urethane).

In another particular embodiment, the at least one elastomer matrix of the porous biomaterial is a matrix comprising an elastomer based on poly(caprolactone-urea-urethane), poly(lactic acid-urea-urethane), poly(glycolic acid-urea-urethane), poly(hydroxyvalerate-urea-urethane) and poly(hydroxybutyrate-urea-urethane).

In another particular embodiment, the at least one elastomer matrix of the porous biomaterial is a matrix comprising an elastomer based on poly(caprolactone-urea-urethane), poly(lactic acid-urea-urethane), poly(glycolic acid-urea-urethane), poly(hydroxyvalerate-urea-urethane), poly(hydroxybutyrate-urea-urethane) and poly(dioxanone-urea-urethane).

In another particular embodiment, the at least one elastomer matrix of the porous biomaterial is a matrix comprising an elastomer based on poly(caprolactone-urea-urethane), poly(lactic acid-urea-urethane), poly(glycolic acid-urea-urethane), poly(hydroxyvalerate-urea-urethane), poly(hydroxybutyrate-urea-urethane), poly(dioxanone-urea-urethane) and poly(ethylene adipate-urea-urethane).

In another particular embodiment, the at least one elastomer matrix of the porous biomaterial is a matrix comprising an elastomer based on poly(caprolactone-urea-urethane), poly(lactic acid-urea-urethane), poly(glycolic acid-urea-urethane), poly(hydroxyvalerate-urea-urethane), poly(hydroxybutyrate-urea-urethane), poly(dioxanone-urea-urethane), poly(ethylene adipate-urea-urethane) and poly(butylene adipate-urea-urethane).

These elastomers indeed allow the implementation of the present invention and furthermore have the advantages of being cytocompatible, of allowing the restoration of the physiological stresses of the deficient tissue, of avoiding a reoperation after restoration and of allowing correct reconstruction of the deficient tissue. Particularly advantageously, the at least one elastomer matrix of the porous biomaterial is a matrix comprising an elastomer based on poly(caprolactone-urea-urethane). This matrix comprising an elastomer based on poly(caprolactone-urea-urethane) has the advantage of having an elastomer nature, providing the matrix with the flexibility and an interconnected porous structure suitable for tissue reconstruction.

In a particular embodiment of the invention, the non-sulfated polysaccharide may be chosen from the group comprising carrageenans, alginates, xanthan, chitosan, chitin, hyaluronic acid, glycogen, cellulose and its derivatives, pectins, starch and its derivatives, dextrans and xylans, or a mixture thereof.

Advantageously, the non-sulfated polysaccharide can therefore consist of a single polysaccharide or of a mixture of non-sulfated polysaccharides.

In a particularly advantageous embodiment of the invention, the non-sulfated polysaccharide according to the invention is hyaluronic acid.

For the purposes of the present invention, the term “hyaluronic acid” means hyaluronic acid, crosslinked or non-crosslinked, alone or as a mixture; optionally chemically modified by substitution, alone or as a mixture; and/or optionally in the form of one of its salts, alone or as a mixture.

Advantageously, hyaluronic acid is a high molecular weight hyaluronic acid.

For the purposes of the present invention, the term “high-molecular-weight hyaluronic acid” means a hyaluronic acid having a molecular weight greater than or equal to 1000 kDa. On the contrary, the term “low molecular weight hyaluronic acid” means hyaluronic acid having a molecular weight of less than 1000 kDa.

In a particular embodiment of the invention, the hyaluronic acid has a molecular weight greater than or equal to 1000 kDa, advantageously greater than or equal to 10,000 kDa, advantageously greater than or equal to 100000 kDa, advantageously greater than or equal to 1,000,000 kDa, advantageously greater than or equal to 1,500,000 kDa, advantageously greater than or equal to 2,000,000 kDa. Advantageously, the hyaluronic acid according to the invention has a molecular weight of 1,500,000 kDa. Advantageously, the use of a high molecular weight hyaluronic acid, in addition to these non-immunogenic and anti-angiogenic properties, allows the structuring of matrix macromolecules and particularly collagens during the early phases of healing, which cannot be obtained with a low molecular weight hyaluronic acid.

In an advantageous embodiment of the invention, the biomaterial according to the invention comprises:
at least one elastomer matrix comprising an elastomer based on poly(ester-urea-urethane), the ester being chosen from caprolactone oligomers (PCL), lactic acid oligomers (PLA), glycolic acid oligomers (PGA), hydroxybutyrate oligomers (PHB), hydroxyvalerate oligomers (PVB), dioxanone oligomers (PDO), poly(ethylene adipate) oligomers (PEA), poly(butylene adipate) oligomers (PBA) or combinations thereof, and
a non-sulfated polysaccharide.

In a first particular embodiment of the invention, the porous biomaterial according to the invention comprises:
at least one elastomer matrix comprising a poly(caprolactone-urea-urethane)-based elastomer, and
a non-sulfated polysaccharide.

In a second particular embodiment of the invention, the porous biomaterial according to the invention comprises:
at least one elastomer matrix comprising a poly(lactic acid-urea-urethane)-based elastomer, and
a non-sulfated polysaccharide.

In a third particular embodiment of the invention, the porous biomaterial according to the invention comprises:
at least one elastomer matrix comprising a poly(glycolic acid-urea-urethane)-based elastomer, and
a non-sulfated polysaccharide.

In a fourth particular embodiment of the invention, the porous biomaterial according to the invention comprises:
at least one elastomer matrix comprising an elastomer based on poly(caprolactone-urea-urethane) and on poly(lactic acid-urea-urethane), and
a non-sulfated polysaccharide.

In a fifth particular embodiment of the invention, the porous biomaterial according to the invention comprises:
at least one elastomer matrix comprising an elastomer based on poly(caprolactone-urea-urethane) and on poly(glycolic acid-urea-urethane), and
a non-sulfated polysaccharide.

In a sixth particular embodiment of the invention, the porous biomaterial according to the invention comprises:
at least one elastomer matrix comprising an elastomer based on poly(lactic acid-urea-urethane) and on poly(glycolic acid-urea-urethane), and
a non-sulfated polysaccharide.

In a seventh particular embodiment of the invention, the porous biomaterial according to the invention comprises:
at least one elastomer matrix comprising an elastomer based on poly(caprolactone-urea-urethane), poly(lactic acid-urea-urethane) and poly(glycolic acid-urea-urethane) and
a non-sulfated polysaccharide.

In an eighth particular embodiment of the invention, the porous biomaterial according to the invention comprises:
at least one elastomer matrix comprising an elastomer based on poly(hydroxybutyrate-urea-urethane), and
a non-sulfated polysaccharide.

In a ninth particular embodiment of the invention, the porous biomaterial according to the invention comprises:
at least one elastomer matrix comprising an elastomer based on poly(hydroxyvalerate-urea-urethane), and
a non-sulfated polysaccharide.

In a tenth particular embodiment of the invention, the porous biomaterial according to the invention
at least one elastomer matrix comprising an elastomer based on poly(dioxanone-urea-urethane), and
a non-sulfated polysaccharide.

In an eleventh particular embodiment of the invention, the porous biomaterial according to the invention comprises:
at least one elastomer matrix comprising an elastomer based on poly(ethylene adipate-urea-urethane), and
a non-sulfated polysaccharide.

In a twelfth particular embodiment of the invention, the porous biomaterial according to the invention comprises:
at least one elastomer matrix comprising an elastomer based on poly(butylene adipate-urea-urethane), and
a non-sulfated polysaccharide.

Advantageously, according to one of the aforementioned embodiments (embodiments 1 to 12), the non-sulfated polysaccharide may be hyaluronic acid.

Advantageously, hyaluronic acid is a high molecular weight hyaluronic acid.

In a particularly advantageous embodiment of the invention, the porous biomaterial according to the invention comprises:
at least one elastomer matrix comprising an elastomer based on poly(caprolactone-urea-urethane), and
hyaluronic acid.

Advantageously, the porous biomaterial comprises:
at least one elastomer matrix comprising an elastomer based on poly(caprolactone-urea-urethane), and
high molecular weight hyaluronic acid.

Advantageously, the porous biomaterial consists solely of:
at least one elastomer matrix comprising an elastomer based on poly(caprolactone-urea-urethane), and
high molecular weight hyaluronic acid.

In an advantageous embodiment of the invention, the biomaterial according to the invention contains:
at least one elastomer matrix comprising an elastomer based on poly(ester-urea-urethane), the ester being chosen from caprolactone oligomers (PCL), lactic acid oligomers (PLA), glycolic acid oligomers (PGA), hydroxybutyrate oligomers (PHB), hydroxyvalerate oligomers (PVB), dioxanone oligomers (PDO), poly(ethylene adipate) oligomers (PEA), poly(butylene adipate) oligomers (PBA) or combinations thereof, and
a non-sulfated polysaccharide.

Advantageously, the porous biomaterial contains:
at least one elastomer matrix comprising an elastomer based on poly(caprolactone-urea-urethane), and
high molecular weight hyaluronic acid.

Advantageously, the inventors have demonstrated that the specific combination of hyaluronic acid, in particular of high molecular weight hyaluronic acid, and of at least one elastomer matrix comprising an elastomer based on poly(caprolactone-urea-urethane), allows an increase in cell migration, but also better vascularization and tissue reconstruction inside and at the periphery of the porous biomaterial with respect to the use of the porous elastomer matrix comprising an elastomer based on poly(caprolactone-urea-urethane) alone.

Indeed, the addition of acid hyaluronic acid leads to an increase in the collagen synthesis, thus making it possible to obtain a more structured tissue.

In a particular embodiment of the invention, the biomaterial has a multiscale pore size of between 50 μm and 2000 μm. For the purposes of the present invention, the terms “pore size” and “pore diameter” may be used interchangeably. By “multi-scale pore size” is meant a variable distribution of pore sizes, that is to say comprising both pores of several microns and pores of smaller sizes, in variable proportions. By way of example, a biomaterial having a multiscale pore size of between 50 μm and 2000 μm means that the biomaterial comprises both and in the same biomaterial pores having variable sizes, of between 50 μm and 2000 μm. By way of non-limiting example, a biomaterial having a multiscale pore size of between 50 μm and 2000 μm means that the biomaterial comprises simultaneously, and in the same biomaterial, pores having for example a size of 50 μm, pores having a size of 100 μm, pores having a size of 500 μm, pores having a size of 1500 μm, and pores having a size of 2000 μm.

Advantageously, the biomaterial has a multiscale pore size of between 50 μm and 1200 μm.

Advantageously, the average pore size is between 500 μm and 700 μm.

Advantageously, said biomaterial has a multiscale pore size of between 500μm and 2000 μm.

In an advantageous embodiment of the invention, the pores of the biomaterial have a rough surface.

In an advantageous embodiment of the invention, the biomaterial has a total porosity greater than or equal to 60%. Within the meaning of the present invention, the term “total porosity” means the ratio of the volume of the empty spaces of material to the overall volume of the biomaterial.

Advantageously, the total porosity of the porous biomaterial is greater than 60%, advantageously greater than 61%, advantageously greater than 62%, advantageously greater than 63%, advantageously greater than 64%, advantageously greater than 65%, advantageously greater than 66%, advantageously greater than 67%, advantageously greater than 68%, advantageously greater than 69%, advantageously greater than 70%, advantageously greater than 71%, advantageously greater than 72%, advantageously greater than 73%, advantageously greater than 74%, advantageously greater than 75%, advantageously greater than 76%, advantageously greater than 77%, advantageously greater than 78%, advantageously greater than 79%, advantageously greater than 80%, advantageously greater than 81%, advantageously greater than 82%, advantageously greater than 83%, advantageously greater than 83%. 892 greater than 84%, advantageously greater than 85%, advantageously greater than 86%, advantageously greater than 87%, advantageously greater than 88%, advantageously greater than 89%, advantageously greater than 90%, advantageously greater than 91%, advantageously greater than 92%, advantageously greater than 93%, advantageously greater than 94%, advantageously greater than 95%, advantageously greater than 96%, advantageously greater than 97%, advantageously greater than 98%, advantageously greater than 99%.

In a particularly advantageous embodiment, the biomaterial has a total porosity of greater than 80%.

Advantageously, the total porosity of the biomaterial is between 60% and 95%, advantageously between 61% and 89%, advantageously between 62% and 88%, advantageously between 63% and 87%, advantageously between 64% and 86%, advantageously between 65% and 85%, advantageously between 66% and 84%, advantageously between 67% and 83%, advantageously between 68% and 82%, advantageously between 69% and 81%, advantageously between 70% and 80%.

In a particularly advantageous embodiment, the porous biomaterial has a total porosity of between 70% and 95%.

In a particular embodiment of the invention, the biomaterial has an interconnectivity between the pores of between 60% and 100%.

Advantageously, the interconnectivity between the pores is between 65% and 100%, advantageously between 70% and 100%, advantageously between 75% and 100%, advantageously between 80% and 100%, advantageously between 85% and 100%, advantageously between 90% and 100%, advantageously between 91% and 100%, advantageously between 92% and 100%, advantageously between 93% and 100%, advantageously between 94% and 100%, advantageously between 95% and 100%, advantageously between 96% and 100%, advantageously between 97% and 100%, advantageously between 98% and 100%, advantageously between 99% and 100%.

In a particularly advantageous embodiment of the invention, the interconnectivity between the pores is greater than 65%, advantageously greater than 70%, advantageously greater than 75%, advantageously greater than 80%, advantageously greater than 85%, advantageously greater than 90%, advantageously greater than 91%, advantageously greater than 92%, advantageously greater than 93%, advantageously greater than 94%, advantageously greater than 95%, advantageously greater than 96%, advantageously greater than 97%, advantageously greater than 98%, advantageously greater than 99%.

In a particularly advantageous embodiment of the invention, the biomaterial has an interconnectivity between the pores of 100%.

In a particularly advantageous embodiment, the biomaterial according to the invention has a pore size of between 50 μm and 2000 μm, a total porosity of greater than or equal to 60% and an interconnectivity between the pores of between 60% and 100%.

Advantageously, the biomaterial according to the invention has a mean pore size of between 50 μm and 1200 μm, a total porosity of between 60% and 95% and an interconnectivity between the pores of between 60% and 100%.

Advantageously, the biomaterial according to the invention has a mean pore size of between 500 μm and 700 μm, a total porosity of between 70% and 95% and an interconnectivity between the pores of 100%.

In a particularly advantageous embodiment, the porous biomaterial comprising at least one elastomer matrix comprising an elastomer based on poly(caprolactone-urea-urethane) and hyaluronic acid, has a pore size of between 500 μm and 2000 μm, a total porosity of between 60% and 95% and an interconnectivity between the pores of between 60% and 100%.

Advantageously, the biomaterial comprising at least one elastomer matrix comprising an elastomer based on poly(caprolactone-urea-urethane) and hyaluronic acid, has an average pore size of between 500 μm and 700 μm, a total porosity of between 70% and 95% and an interconnectivity between the pores of 100%. The porosity of the material, the pore size and their interconnection have a major influence on the ability of the biomaterial to vascularize and resorb progressively.

Thus, by virtue of its total porosity of between 60% and 95% and its pore size of between 500 μm and 2000 μm and its interconnectivity between the pores of 100%, the biomaterial comprising at least one elastomer matrix comprising an elastomer based on poly(caprolactone-urea-urethane) and hyaluronic acid is particularly suitable for the adhesion and migration of the cells of the connective tissue and of the blood vessels. Indeed, the interconnected porous network makes it possible to guide the attachment and growth of the cells, and therefore the growth of the newly formed connective tissue. In addition, the presence of hyaluronic acid stimulates angiogenesis, thus making it possible to improve revascularization and integration of the biomaterial. At the same time, the fibroblasts adhere and proliferate inside and at the periphery of the biomaterial. The resorption of the biomaterial and the simultaneous production of collagen by the fibroblasts present inside and at the periphery of the biomaterial lead to its complete substitution by the newly formed connective tissue after a few months.

Thus, the biomaterial comprising at least one elastomer matrix comprising an elastomer based on poly(caprolactone-urea-urethane) and hyaluronic acid promotes revascularization, rapid integration of soft tissues and offers a safe alternative to autologous connective tissue.

The size of the biomaterial according to the invention depends on the size and thickness of the tissue defect. In a particular embodiment of the invention, the biomaterial has a size of between 5 mm and 20 cm and a thickness of between 100 μm and 4 cm.

Advantageously, the size of the biomaterial is between 5 mm and 20 cm, advantageously between 10 mm and 20 cm, advantageously between 50 mm and 20 cm, advantageously between 100 mm and 20 cm, advantageously between 500 mm and 20 cm, advantageously between 1 cm and 20 cm, advantageously between 2 cm and 20 cm, advantageously between 3 cm and 20 cm, advantageously between 4 cm and 20 cm, advantageously between 5 cm and 20 cm, advantageously between 6 cm and 20 cm, advantageously between 7 cm and 20 cm, advantageously between 8 cm and 20 cm, advantageously between 9 cm and 20 cm, advantageously between 10 cm and 20 cm, advantageously between 11 cm and 20 cm, advantageously between 12 cm and 20 cm, advantageously between 13 cm and 20 cm, advantageously between 14 cm and 20 cm, advantageously between 15 cm and 20 cm.

Advantageously, the thickness of the biomaterial is between 100 μm and 4 cm, advantageously between 200 μm and 4 cm, advantageously between 500 μm and 4 cm, advantageously between 1 mm and 4 cm, advantageously between 2 mm and 4 cm, advantageously between 3 mm and 4 cm, advantageously between 4 mm and 4 cm, advantageously between 5 mm and 4 cm, advantageously between 6 mm and 4 cm, advantageously between 7 mm and 4 cm, advantageously between 8 mm and 4 cm, advantageously between 9 mm and 4 cm, advantageously between 1 cm and 4 cm, advantageously between 1 cm and 3 cm.

In a particular advantageous embodiment, the thickness of the biomaterial is between 1 and 3 mm, when the biomaterial according to the invention is used in the reinforcement, reconstruction and/or filling in of tissue defects of the mucosa, and in particular of the gum.

In a particular advantageous embodiment, the surface of the biomaterial is at least 25 mm².

Advantageously, the biomaterial has a surface area of at least 50 mm², advantageously at least 100 mm², advantageously at least 150 mm², advantageously at least 200 mm², advantageously at least 250 mm², advantageously at least 300 mm², advantageously at least 350 mm², advantageously at least 400 mm², advantageously at least 450 mm², advantageously at least 500 mm², advantageously at least 550 mm², advantageously at least 600 mm², advantageously at least 650 mm², advantageously at least 700 mm², advantageously at least 750 mm², advantageously at least 800 mm², advantageously at least 850 mm², advantageously at least 900 mm², advantageously at least 950 mm², advantageously at least 1000 mm², advantageously at least 15 cm², advantageously at least 20 cm², advantageously at least 25 cm², advantageously at least 25 cm², advantageously, at least 30 cm², advantageously at least 35 cm², advantageously at least 40 cm², advantageously at least 45 cm², advantageously at least 50 cm, advantageously at least 55 cm², advantageously, at least 60 cm², advantageously at least 65 cm², advantageously at least 70 cm², advantageously, at least 75 cm², advantageously at least 80 cm², advantageously at least 85 cm², advantageously at least 90 cm², advantageously at least 95 cm², advantageously at least 100 cm², advantageously at least 150 cm², advantageously at least 200 cm², advantageously at least 250 cm², advantageously, at least 300 cm², advantageously at least 350 cm², advantageously at least 400 cm². In an advantageous embodiment, the biomaterial has a volume of between 25 mm² and 400 cm².

In a particular embodiment of the invention, the biomaterial has a volume of at least 1 mm³.

Advantageously, the biomaterial has a volume of at least 2 mm³, advantageously at least 3 mm³, advantageously at least 4 mm³, advantageously at least 5 mm³, advantageously at least 6 mm³, advantageously at least 7 mm³, advantageously at least 8 mm³, advantageously at least 9 mm³, advantageously at least 10 mm³, advantageously at least 20 mm³, advantageously at least 30 mm³, advantageously at least 40 mm³, advantageously at least 50 cm³, advantageously at least 60 mm³, advantageously at least 70 mm³, advantageously at least 80 mm³, advantageously at least 90 mm³, advantageously at least 100 mm³, advantageously at least 150 mm³, advantageously at least 200 mm³, advantageously at least 250 mm³, advantageously at least 300 mm³, advantageously at least 350 mm³, advantageously at least 400 mm³, advantageously at least 450 mm³, advantageously at least 500 mm³, advantageously at least 550 mm³, advantageously at least 600 mm³, advantageously at least 650 mm³, advantageously at least 700 mm³, advantageously at least 700 mm³, advantageously at least 750 mm³, advantageously at least 800 mm³, advantageously at least 850 mm³, advantageously at least 900 mm³, advantageously at least 950 mm³, advantageously at least 1 cm³, advantageously at least 1.5 cm³, advantageously at least 2 cm³, advantageously at least 2.5 cm³, advantageously at least 3 cm³, advantageously at least 3.5 cm³, advantageously at least 4 cm³, advantageously at least 4.5 cm³, advantageously at least 5 cm³, advantageously at least 5.5 cm³, advantageously at least 6 cm³, advantageously at least 6.5 cm³, advantageously at least 7 cm³, advantageously at least 7.5 cm³, advantageously at least 8 cm³, advantageously at least 8.5 cm³, advantageously at least 9 cm³, advantageously at least 9.5 cm³, advantageously at least 10 cm³. In an advantageous embodiment, the biomaterial has a volume of between 1 mm³and 10 cm³.

In an advantageous embodiment of the invention, the biomaterial according to the invention may be in the form of a sponge, a film, a membrane, granules, monoliths or a wound dressing.

In a particular embodiment of the invention, the biomaterial according to the invention is used alone. In another embodiment of the invention, the biomaterial may further be used in combination with an active agent. Advantageously, the active agent is arranged inside the pores of the biomaterial according to the invention, partially or completely covering the pores of the biomaterial. Advantageously, the active agent may be added by one of the following methods: covering the biomaterial with the active agent, immersing the biomaterial in the active agent, spraying the active agent on the biomaterial, vaporizing the active agent on the biomaterial, or any other technique well known to a person skilled in the art making it possible to fill and/or fill in the pores of said biomaterial. Advantageously, the active agent may be any therapeutic or pharmaceutically active agent (including, but not limited to, nucleic acids, proteins, lipids, and carbohydrates) that has desirable physiological characteristics for application to the site of implantation. Therapeutic agents include, but are not limited to, anti-infectious agents such as antibiotics and antiviral agents; chemotherapeutic agents (e.g., anticancer agents); anti-rejection agents; analgesics and analgesic combinations; anti-inflammatory agents; hormones such as steroids; growth factors (including, but not limited to, cytokines, chemokines and interleukins), coagulation factors (factors VII, VIII, IX, X, XI, XII, V,) , albumin, fibrinogen, von Willebrand factor, thrombin inhibitors, antithrombogenic agents, thrombolytic agents, fibrinolytic agents, vasospasm inhibitors, calcium channel inhibitors, vasodilators, anti-hypertensive agents, antimicrobial agents, antibiotics, antibiotics, surface glycoprotein receptor inhibitors, antiplatelet agents, antimitotic drugs, microtubule inhibitors, antiplatelet agents, antimitotic drugs, microtubule inhibitors, actin inhibitors, antisecretory agents, remodelling inhibitors, antisense nucleotides, anti metabolites, antiproliferative agents, anticancer chemotherapeutic agents, anti-inflammatory steroids, non-steroidal anti-inflammatory agents, immunosuppressive agents, growth hormone antagonists, growth factors, dopamine agonists, radiotherapeutic agents, peptides, proteins, enzymes, extracellular matrix components, angiotensin converting enzyme (ACE) inhibitors, free radical scavengers, chelators, antioxidants, antipolymerases, antiviral agents, photodynamic therapy agents and gene therapy agents and other naturally occurring or genetically modified proteins, polysaccharides, glycoproteins and lipoproteins or a combination thereof, the list not being limiting. In a particularly advantageous embodiment of the invention, the active agent is a combination of therapeutic agents, and in particular a combination of antibiotics and growth factors.

Another aspect of the invention relates to the biomaterial according to the invention for use in reinforcing, reconstructing and/or filling in tissue defects. Within the meaning of the present invention, the term “strengthening tissue defects” is intended to mean the increase in tissue density by induction of collagen synthesis and/or collagen deposition, due to the biocompatibility of the biomaterial, and in particular thanks to the presence of hyaluronic acid.

Within the meaning of the present invention, the term “reconstruction of tissue defects” means the repair of tissue defects by induction of collagen synthesis and/or deposition of collagen, due to the biocompatibility of the biomaterial, and in particular thanks to the presence of hyaluronic acid.

Within the meaning of the present invention, the term “fill of tissue defects” is intended to mean filling in of tissue defects by induction of collagen synthesis and/or collagen deposition, due to the biocompatibility of the biomaterial, and in particular thanks to the presence of hyaluronic acid.

In a particular embodiment of the invention, the tissue reinforcement, reconstruction and/or filling in is greater than or equal to 5% by volume of the volume of the tissue defect to be reinforced, reconstructed and/or filling. Advantageously, the reinforcement, reconstruction and/or tissue filling in is greater than or equal to 6% by volume of the tissue. The volume of tissue to be reconstructed and/or filled in is advantageously greater than or equal to 7%, advantageously greater than or equal to 8%, Advantageously greater than or equal to 9%, advantageously greater than or equal to 10%, advantageously greater than or equal to 11%, Advantageously greater than or equal to 12%, advantageously greater than or equal to 13%, advantageously greater than or equal to 14%, Advantageously greater than or equal to 15%, advantageously greater than or equal to 16%, advantageously greater than or equal to 17%, Advantageously greater than or equal to 18%, advantageously greater than or equal to 19%, advantageously greater than or equal to 20%, Advantageously greater than or equal to 21%, advantageously greater than or equal to 22%, advantageously greater than or equal to 23%, Advantageously, greater than or equal to 24%, advantageously greater than or equal to 25%, advantageously greater than or equal to 26%, Advantageously greater than or equal to 27%, advantageously greater than or equal to 28%, advantageously greater than or equal to 29%, Advantageously greater than or equal to 30%, advantageously greater than or equal to 31%, advantageously greater than or equal to 32%, Advantageously greater than or equal to 33%, advantageously greater than or equal to 34%, advantageously greater than or equal to 35%, Advantageously greater than or equal to 36%, advantageously greater than or equal to 37%, advantageously greater than or equal to 38%, Advantageously greater than or equal to 39%, advantageously greater than or equal to 40%, advantageously greater than or equal to 41%, Advantageously greater than or equal to 42%, advantageously greater than or equal to 43%, advantageously greater than or equal to 44%, Advantageously greater than or equal to 45%, advantageously greater than or equal to 46%, advantageously greater than or equal to 47%, Advantageously greater than or equal to 48%, advantageously greater than or equal to 49%. Advantageously, the reinforcement, reconstruction and/or tissue filling in is less than or equal to 50% by volume of the volume of the tissue defect to be reinforced, reconstructed and/or filled in.

In a particularly advantageous embodiment of the invention, the porous biomaterial according to the invention can be used for reinforcement, reconstruction and/or tissue filling in in human beings or animals. By way of example, the animal may be a horse, a pony, a dog, a cat, a rat, a mouse, a pig, a sow, a cow, a beef, a bull, a calf, a goat, a sheep, a ram, an agnella, a lamb, a donkey, a camel, a dromedary, the list not being limiting.

Advantageously, the porous biomaterial according to the invention can be used for reinforcement, reconstruction and/or filling in defects of soft tissues and/or epithelial tissues.

Within the meaning of the present invention, the term “soft tissues” is intended to mean tissues, non-osseous and not composed of epithelium, which surround, support and connect the organs and other tissues.

Advantageously, the soft tissue surrounds, supports and connects the organs and other body parts; confer shape and structure to the body; protects the organs; circulates liquids, such as blood, from one body part to another; stores energy.

In a particular embodiment of the invention, the biomaterial according to the invention can be used to reinforce, reconstruct and/or fill in any type of soft tissue, of human or animal origin.

Advantageously, the soft tissue may be chosen from the group comprising: fibrous tissue, muscles, in particular smooth muscles, skeletal muscles and cardiac muscle, synovial tissue, blood vessels, lymphatic vessels, viscera and nerves, the list not being limiting.

In a particular embodiment, the biomaterial according to the invention can be used to reinforce, reconstruct and/or fill in any type of epithelial tissues, of human or animal origin. Advantageously, the biomaterial according to the invention can be used for strengthening, reconstructing and/or filling defects in the skin and/or mucous membranes. Advantageously, the mucosa may be an oral mucosa.

In a particular embodiment, the biomaterial according to the invention can be used to reinforce, reconstruct and/or fill in any type of epithelial tissues, advantageously in the reinforcement, reconstruction and/or gum filling in, in particular to obtain root coverage, for treating of gum recessions, thickening of the tissues so as to obtain a thick biotype, increasing the keratinized gum band, restoring the support and anchoring of the teeth, or reconstructing the tissues following periodontitis.

In a particular embodiment, the biomaterial according to the invention can be used to reinforce the defects of soft tissues and/or epithelial tissues, in particular in the context of a gynaecological, urinary or visceral (or parietal) surgery, such as, for example, to reinforce a vascular wound, a digestive wound, or an eventration. In another embodiment, the biomaterial according to the invention can be used for the design of reinforcing implants for treatment of pelvic organs prolapse, more particularly in the treatment of pelvic organ prolapse of women: anterior stage (urinary, cystocele, stress incontinence), medium stage (genital, colpocele) and/or posterior stage (digestive rectocele).

Another aspect of the invention relates to the biomaterial according to the invention for use in the treatment of burns. Advantageously, the biomaterial according to the invention is particularly useful for treating burns.

Advantageously, the biomaterial according to the invention is particularly useful for the treatment of thermal burns, cold burns, electric burns, chemical burns and radiological burns and photochemical burns.

Within the meaning of the present invention, “thermal burns” means external thermal burns caused by external contact with a flame, hot vapours or boiling liquids, or by contact (the severity then depends on the temperature of the object and on the contact time) and internal thermal burns concern the respiratory or digestive tract and result from the absorption or the inhalation of a hot product (food, a gas, inter alia the gases produced by combustion) or a caustic substance (chemical product).

For the purposes of the present invention, “cold burns” means frostbite. Frostbite may be caused by something cold and friction.

For the purposes of the present invention, the term “electrical burns” is intended to mean partial or total destruction which may relate to the skin, mucous membranes (optionally internal), soft parts of the tissues due to an electric arc (thermal burns by conflagration) or by direct contact with the conductor (always deep).

For the purposes of the present invention, the term “chemical burns” is intended to mean partial or total destruction which may relate to the skin, mucous membranes (optionally internal), soft parts of the tissues due to the caustic action of a strong acid (hydrochloric acid, sulfuric acid, nitric acid) or of a strong base (sodium hydroxide, potassium hydroxide).

For the purposes of the present invention, the term “radiological burns” means burns or radiodermites caused by electromagnetic radiation, by corpuscular bodies.

In a particularly advantageous embodiment of the invention, the biomaterial according to the invention can be used for the treatment of burns in human beings or animals. By way of example, the animal may be a horse, a pony, a dog, a cat, a rat, a mouse, a pig, a sow, a cow, a bull, a beef, a calf, a goat, a sheep, a ram, an agnella, a lamb, a donkey, a camel, a dromedary, the list not being limiting.

Another aspect of the invention relates to a method for preparing a biomaterial according to the invention. In a particular embodiment of the invention, the biomaterial according to the invention is obtained by the poly-HIPE method (formation and polymerization/crosslinking of high internal phase emulsions). The high internal phase emulsions or HIPE consist of immiscible liquid/liquid dispersed systems, in which the volume of the internal phase, also called dispersed phase, occupies a volume greater than about 74%-75% of the total volume of the emulsion, that is to say a volume greater than that which is geometrically possible for the compact packing of monodisperse spheres.

In a particular embodiment, the method for preparing the biomaterial comprises the following steps:
a) preparing an organic phase comprising the compounds necessary for the synthesis of the poly(ester-urea-urethane),
b) solubilizing the non-sulfated polysaccharide in an aqueous liquid phase and then adding the solubilized non-sulfated polysaccharide into the organic phase of step a) to form an emulsion,
c) polymerizing/crosslinking the emulsion obtained at step b) to obtain said biomaterial, and
d) washing said biomaterial obtained at step c),
e) drying said biomaterial obtained in step d).

In one embodiment of the invention, step a) consists in preparing an organic phase comprising the compounds necessary for the synthesis of the poly(ester-urea-urethane). Advantageously, the organic phase also comprises an oligoester, an organic solvent, a crosslinking agent, a catalyst and a surfactant.

Advantageously, the organic phase comprises an organic solvent, the polycaprolactone triol oligomer, the Span80 surfactant, the hexamethylene diisocyanate crosslinking agent (HMDI) and the dibutyltin dilaurate catalyst (DBTDL). Advantageously, the organic solvent is toluene.

In a particular embodiment, step a) comprises a first step a1) consisting in solubilizing the polycaprolactonetriol oligomer and the Span80 surfactant in the organic solvent, then a second step a2) consisting in adding the crosslinking agent HMDI and the DBTDL catalyst to the solution of step a1) to form the organic phase. In an advantageous embodiment of the invention, 2.4 mL of organic solvent is used, 1.3 g of polycaprolactonetriol oligomer, 1.3 g of Span 80 surfactant, 1.04 mL of HMDI crosslinking agent and 12 drops of DBTDL catalyst are used. Advantageously, a person skilled in the art will know how to adapt the amounts of toluene, of polycaprolactone triol oligomer, of Span80 surfactant, of HMDI crosslinking agent and of DBTDL catalyst according to the desired pore size for the porous biomaterial according to the invention. Advantageously, the organic solvent is toluene.

In a particular embodiment, step b) of the process consists in solubilizing the non-sulfated polysaccharide in an aqueous liquid phase and then adding the solubilized non-sulfated polysaccharide to the organic phase of step a) to form an emulsion. In a particular embodiment of the invention, the non-sulfated polysaccharide must have been dissolved in an aqueous liquid phase. In a particular embodiment of the invention, the aqueous liquid phase is sterilized distilled water. Advantageously, a person skilled in the art will know how to adapt the amount of water according to the desired size of the pores for the biomaterial. In an advantageous embodiment of the invention, the amount of distilled water used is 50 mL.

In a particular embodiment of the invention, the aqueous liquid phase is gradually poured into the organic phase while stirring, until an emulsion is obtained.

Advantageously, the non-sulfated polysaccharide is introduced at a concentration of at least 0.5 mg/mL, advantageously at a concentration of at least 1.0 mg/mL, advantageously at a concentration of at least 1.5 mg/mL, advantageously at a concentration of at least 2.0 mg/mL, advantageously at a concentration of at least 2.5 mg/mL, advantageously at a concentration of at least 3.0 mg/mL, advantageously at a concentration of at least 3.5 mg/mL, advantageously at a concentration of at least 4.0 mg/mL, advantageously at a concentration of at least 4.5 mg/mL, advantageously at a concentration of at least 5.0 mg/mL, advantageously at a concentration of at least 5.5 mg/mL, advantageously at a concentration of at least 6.0 mg/mL, advantageously at a concentration of at least 6.5 mg/mL, advantageously at a concentration of at least 7.0 mg/mL, advantageously at a concentration of at least 7.5 mg/mL, advantageously at a concentration of at least 8.0 mg/mL, advantageously at a concentration of at least 8.5 mg/mL, advantageously at a concentration of at least 9.0 mg/mL, advantageously at a concentration of at least 9.5 mg/mL, advantageously at a concentration of at least 10.0 mg/mL, advantageously at a concentration of at least 10.5 mg/mL, advantageously at a concentration of at least 11.0 mg/mL, advantageously at a concentration of at least 11.5 mg/mL, advantageously at a concentration of at least 12.0 mg/mL, advantageously at a concentration of at least 12.5 mg/mL, advantageously at a concentration of at least 13.0 mg/mL, advantageously at a concentration of at least 13.5 mg/mL, advantageously at a concentration of at least 14.0 mg/mL, advantageously at a concentration of at least 14.5 mg/ml, advantageously at a concentration of at least 15.0 mg/mL, advantageously at a concentration of at least 15.5 mg/mL, advantageously at a concentration of at least 16.0 mg/mL, advantageously at a concentration of at least 16.5 mg/mL, advantageously, at a concentration of at least 17.0 mg/mL, advantageously at a concentration of at least 17.5 mg/mL, advantageously at a concentration of at least 18.0 mg/mL, advantageously at a concentration of at least 18.5 mg/mL, advantageously at a concentration of at least 19.0 mg/mL, advantageously at a concentration of at least 19.5 mg/mL, advantageously at a concentration of at least 20.0 mg/mL. Advantageously, the non-sulfated polysaccharide is introduced at a concentration of between 0.5 mg/mL and 20 mg/mL.

In a particular embodiment, the polysaccharide is hyaluronic acid, advantageously high molecular weight hyaluronic acid. Advantageously, the hyaluronic acid is introduced at a concentration of at least 0.5 mg/mL, advantageously at a concentration of at least 1.0 mg/mL, advantageously at a concentration of at least 1.5 mg/mL, advantageously at a concentration of at least 2.0 mg/mL, advantageously at a concentration of at least 2.5 mg/mL, advantageously at a concentration of at least 3.0 mg/mL, advantageously at a concentration of at least 3.5 mg/mL, advantageously at a concentration of at least 4.0 mg/mL, advantageously at a concentration of at least 4.5 mg/mL, advantageously at a concentration of at least 5.0 mg/mL, advantageously at a concentration of at least 5.5 mg/mL, advantageously at a concentration of at least 6.0 mg/mL, advantageously at a concentration of at least 6.5 mg/mL, advantageously at a concentration of at least 7.0 mg/mL, advantageously at a concentration of at least 7.5 mg/mL, advantageously at a concentration of at least 8.0 mg/mL, advantageously at a concentration of at least 8.5 mg/mL, advantageously at a concentration of at least 9.0 mg/mL, advantageously at a concentration of at least 9.5 mg/mL, advantageously at a concentration of at least 10.0 mg/mL, advantageously at a concentration of at least 10.5 mg/mL, advantageously at a concentration of at least 11.0 mg/mL, advantageously at a concentration of at least 11.5 mg/mL, advantageously at a concentration of at least 12.0 mg/mL, advantageously at a concentration of at least 12.5 mg/mL, advantageously at a concentration of at least 13.0 mg/mL, advantageously at a concentration of at least 13.5 mg/mL, advantageously at a concentration of at least 14.0 mg/mL, advantageously at a concentration of at least 14.5 mg/ml, advantageously at a concentration of at least 15.0 mg/mL, advantageously at a concentration of at least 15.5 mg/mL, advantageously at a concentration of at least 16.0 mg/mL, advantageously at a concentration of at least 16.5 mg/mL, advantageously, at a concentration of at least 17.0 mg/mL, advantageously at a concentration of at least 17.5 mg/mL, advantageously at a concentration of at least 18.0 mg/mL, advantageously at a concentration of at least 18.5 mg/mL, advantageously at a concentration of at least 19.0 mg/mL, advantageously at a concentration of at least 19.5 mg/mL, advantageously at a concentration of at least 20.0 mg/mL.

In a particular embodiment of the invention, the amount of non-sulfated polysaccharide represents between 0.05% and 2.0% by weight (w/w) relative to the weight of the aqueous liquid phase present in the emulsion. Advantageously, the non-sulfated polysaccharide represents at least 0.05% by weight relative to the weight of aqueous liquid phase present in the emulsion, advantageously at least 0.06%, advantageously at least 0.07%, advantageously at least 0.08%, advantageously at least 0.09%, advantageously at least 0.10%, advantageously at least 0.20%, advantageously at least 0.30%, advantageously at least 0.40%, advantageously at least 0.50%, advantageously at least 0.60%, advantageously at least 0.70%, advantageously at least 0.80%, advantageously at least 0.90%, advantageously at least 1.0%, advantageously at least 11.0%, advantageously at least 1.20%, advantageously at least 1.30%, advantageously at least 1.40%, advantageously at least 1.50%, advantageously at least 1.60%, advantageously at least 1.70%, advantageously at least 1.80%, advantageously at least 1.90%, advantageously at least 2.0% by weight (w/w) relative to the weight of aqueous phase present in the emulsion.

Advantageously, the amount of non-sulfated polysaccharide represents between 0.05% and 2.0% by weight (w/w) relative to the mass of aqueous phase present in the emulsion. Advantageously, the non-sulfated polysaccharide represents between 0.06% and 2.0%, advantageously between 0.07% and 2, 0%, advantageously between 0.08% and 2,0%, advantageously between 0.09% and 2,0%, advantageously between 0.10% and 2%, advantageously between 0.20% and 2.0%, advantageously between 0.30 % and 2.0%, advantageously between 0.40% and 2.0%, advantageously between 0.50% and 2.0%, advantageously between 0.60% and 2.0%, advantageously between 0.70% and 2.0%, advantageously between 0.80% and 2.0%, advantageously between 0.90% and 2.0%, advantageously between 1.0% and 2.0%, advantageously between 1.10% and 2.0%, advantageously between 1.20% and 2.0%, advantageously between 1.30% and 2.0%, advantageously between 1.40% and 2.0%, advantageously between 1.50% and 2.0%, advantageously between 1.60% and 2.0%, advantageously between 1.70% and 2.0%, advantageously between 1.80% and 2.0%, advantageously between 1.90% and 2.0%, by weight (w/w) relative to the mass of aqueous phase present in the emulsion. In a particularly advantageous embodiment of the invention, the non-sulfated polysaccharide represents 0.10 % by weight (w/w) relative to the mass of aqueous liquid phase present in the emulsion.

In a particular embodiment of the invention, the amount of hyaluronic acid represents between 0.05% and 2.0% by weight (w/w) relative to the weight of the aqueous liquid phase present in the emulsion.

Advantageously, the hyaluronic acid represents at least 0.05% by weight relative to the weight of aqueous liquid phase present in the emulsion, advantageously at least 0.06%, advantageously at least 0.07%, advantageously at least 0.08%, advantageously at least 0.09%, advantageously at least 0.10%, advantageously at least 0.20%, advantageously at least 0.30%, advantageously at least 0.40%, advantageously at least 0.50%, advantageously at least 0.60%, advantageously at least 0.70%, advantageously at least 0.80%, advantageously at least 0.90%, advantageously at least 1.0%, advantageously at least 11.0%, advantageously at least 1.20%, advantageously at least 1.30%, advantageously at least 1.40%, advantageously at least 1.50%, advantageously at least 1.60%, advantageously at least 1.70%, advantageously at least 1.80%, advantageously at least 1.90%, advantageously at least 2.0% by weight (w/w) relative to the weight of aqueous phase present in the emulsion.

Advantageously, the amount of hyaluronic acid represents represents between 0.05% and 2.0% by weight (w/w) relative to the mass of aqueous phase present in the emulsion. Advantageously, the non-sulfated polysaccharide represents between 0.06% and 2.0%, advantageously between 0.07% and 2, 0%, advantageously between 0.08% and 2,0%, advantageously between 0.09% and 2,0%, advantageously between 0.10% and 2%, advantageously between 0.20% and 2.0%, advantageously between 0.30 % and 2.0%, advantageously between 0.40% and 2.0%, advantageously between 0.50% and 2.0%, advantageously between 0.60% and 2.0%, advantageously between 0.70% and 2.0%, advantageously between 0.80% and 2.0%, advantageously between 0.90% and 2.0%, advantageously between 1.0% and 2.0%, advantageously between 1.10% and 2.0%, advantageously between 1.20% and 2.0%, advantageously between 1.30% and 2.0%, advantageously between 1.40% and 2.0%, advantageously between 1.50% and 2.0%, advantageously between 1.60% and 2.0%, advantageously between 1.70% and 2.0%, advantageously between 1.80% and 2.0%, advantageously between 1.90% and 2.0%, by weight (w/w) relative to the mass of aqueous phase present in the emulsion. In a particularly advantageous embodiment of the invention, the hyaluronic acid represents 0.10 % by weight (w/w) relative to the mass of aqueous liquid phase present in the emulsion.

In a particular embodiment, step c) of the method consists in polymerizing/crosslinking the emulsion obtained at step b) in order to obtain said biomaterial according to the invention.

Advantageously, the crosslinking is carried out in a mold in order to confer on the biomaterial the desired shape. Advantageously, the emulsion obtained at step b) is placed at a temperature of between 30° C. and 80° C. for 10 to 30 hours. Advantageously, the emulsion obtained in step b) is placed at a temperature of between 35° C. and 65° C., advantageously at a temperature of between 40° C. and 60° C., advantageously at a temperature of between 45° C. and 65° C., advantageously at a temperature of between 50° C. and 60° C., advantageously at a temperature of 55° C. Advantageously, the emulsion obtained at step b) is placed at a temperature of between 30° C. and 70° C. for 10 to 30 hours, advantageously for 11 to 29 hours, advantageously for 12 to 29 hours, advantageously for 13 to 28 hours, advantageously for 14 to 27 hours, advantageously for 15 to 27 hours, advantageously for 16 to 27 hours, advantageously for 17 to 27 hours, advantageously for 18 to 26 hours, advantageously for 19 to 25 hours, advantageously for 20 to 24 hours, advantageously for 22 hours. Advantageously, a person skilled in the art will know how to adapt the temperature according to the desired size of the pores for the biomaterial.

In a particular embodiment of the invention, the biomaterial according to the invention obtained at step c) is annealed prior to step d). Advantageously, the biomaterial according to the invention obtained at step c) is annealed for at least 1 hour at a temperature of at least 50° C. Advantageously, the porous biomaterial according to the invention obtained at step c) is annealed for 2 hours at a temperature of 100° C.

In a particular embodiment, the washing step of step d) makes it possible to remove the reagents necessary for the synthesis of the poly(ester-urea-urethane) which have not reacted during the crosslinking as well as the surfactant and the catalyst still present. Advantageously, the washing of step d) is carried out using one of the following products: dichloromethane, dichloromethane/hexane, hexane, water, a mixture of these products or the successive application of these products. Advantageously, the washing of step d) is carried out by contacting the dried porous biomaterial according to the invention with dichloromethane for at least 24 hours, followed by a washing step with dichloromethane/hexane (50% vol/50% vol) for at least 24 hours, followed by a washing step with hexane for at least 24 hours, and then a last washing with distilled water for at least 24 hours.

In a particular embodiment, the method according to the invention may further comprise a drying step between step c) and step d). Advantageously, this drying step can be carried out by drying in open air or in an oven. A person skilled in the art will know how to adjust the temperature of the oven as a function of the material to be dried. Advantageously, the drying is carried out by open air drying for at least 7 days.

In a particular embodiment, the drying of step e) may be carried out by open air drying or in an oven. A person skilled in the art will know how to adjust the temperature of the oven as a function of the material to be dried. Advantageously, the drying is carried out by open air drying for at least 15 days

In a particular embodiment, the method according to the invention may further comprise a sterilization step f) after the step e) of drying said biomaterial. In a particular embodiment, the sterilization step f) can be carried out directly on the dry biomaterial or after a vacuum washing of the biomaterial in an aqueous medium. Advantageously, the sterilization is carried out after a vacuum washing in an aqueous medium.

In one embodiment, the sterilization step f) can be carried out as follows:
f1) placing the biomaterial according to the invention in sterile water for one hour under vacuum,
f2) replacing the sterile water and placing the biomaterial according to the invention in replacing sterile water for 4 hours under vacuum,
f3) placing the biomaterial according to the invention resulting from step e2) in 70% ethanol for 1 hour under vacuum,
f4) replacing the 70% ethanol with sterile water and placing the biomaterial according to the invention resulting from step e3) in sterile water overnight at ambient pressure,
f5) sterilizing the biomaterial according to the invention resulting from step f4) in water in an autoclave.

In another embodiment, the sterilization step f) can be carried out by gamma radiation.

In another embodiment, the sterilization step f) can be carried out by beta radiation.

Advantageously, the dose of beta and/or gamma radiation may be between 15 and 45 kGy.

Advantageously, the dose of beta and/or gamma radiation is 25 kGy. Advantageously, the dose of beta and/or gamma radiation is 15 kGy.

In another embodiment, the sterilization step f) can be carried out by bringing the biomaterial into contact with ethylene oxide.

In another embodiment, the sterilization step f) can be carried out by bringing the biomaterial into contact with a plasma phase derived from a gas.

In another embodiment, the sterilization step f) can be carried out by irradiating the biomaterial with an electron beam (E-beam, Faisceau E).

The electron beam irradiation treatment has the following advantages: shorter treatment duration, improvement of the efficiency of the supply line, less risk of weakening of the elastomer matrix, less oxidative damages in the biomaterial, absence of colour change of the elastomer matrix, making it clean and safe. In addition, the electron beam irradiation treatment is an ecological treatment.

In a particular embodiment, the method according to the invention may further comprise a step g) of preserving said biomaterial after the sterilization step f). Advantageously, step g) of preserving said biomaterial is carried out by bringing the biomaterial into contact with 70% ethanol until it is used.

In a particular embodiment of the invention, the method for preparing a biomaterial comprising the following steps:
a) preparing an organic phase comprising the required compounds for the synthesis of poly(ester-urea-urethane),
b) solubilizing the non-sulfated polysaccharide in an aqueous liquid phase then adding the solubilized non-sulfated polysaccharide into the organic phase of step a) to form an emulsion,
c) polymerizing/crosslinking the emulsion obtained at step b) to obtain said porous biomaterial, and
d) washing said porous biomaterial obtained at step c),
e) drying the said biomaterial obtained at step d)
f) sterilizing the biomaterial resulting from step d), and
g) optionally, storing the biomaterial.

In a particularly advantageous embodiment of the invention, the process for preparing a biomaterial according to the invention comprising the following steps:
a) preparing an organic phase comprising the compounds necessary for the synthesis of poly(ester-urea-urethane), said step a) comprising a first step a1) consisting in solubilizing, in the organic solvent, the polycaprolactonetriol oligomer and the Span80 surfactant, then a second step a2) consisting in adding the crosslinking agent HMDI and the DBTDL catalyst to the solution of step a1) to form the organic phase,
b) solubilizing the non-sulfated polysaccharide in an aqueous liquid phase based on sterilized distilled water and then adding the non-sulfated polysaccharide solubilized in the organic phase to the liquid of step a) to form an emulsion,
c) polymerizing/crosslinking the emulsion obtained at step b) in order to obtain said porous biomaterial, and
d) washing said biomaterial obtained at step c),
e) drying said biomaterial obtained at step d) for at least 15 days,
f) sterilizing the porous biomaterial resulting from step e), and,
g) optionally, storing the biomaterial.

In a particularly advantageous embodiment of the invention, the method for preparing a biomaterial according to the invention comprising the following steps:
a) preparing an organic phase comprising the compounds necessary for the synthesis of poly(ester-urea-urethane), said step a) comprising a first step a1) consisting in solubilizing in toluene, the polycaprolactone triol oligomer and the surfactant Span80, then a second step a2) consisting in adding the crosslinking agent HMDI and the catalyst DBTDL to the solution of step a1) to form the organic phase,
b) solubilizing the hyaluronic acid, advantageously high molecular weight hyaluronic acid, in an aqueous liquid phase based on sterilized distilled water and then add the non-sulfated polysaccharide dissolved in the organic phase to the liquid of step a) to form an emulsion,
c) polymerize/crosslink the emulsion obtained at step b) to obtain said porous biomaterial, and
d) wash said biomaterial obtained at step c),
e) drying said biomaterial obtained at step d) for at least 15 days,
f) sterilizing the porous biomaterial resulting from step e), and,
g) optionally, storing the biomaterial.

### FIGURES

FIG. 1 represents the porous biomaterial according to the invention. Images were obtained by 3D microscopy (VHX Keyence).

FIG. 2 shows the Fourier Transform InfraRed spectroscopy (FTIR) analysis of the hyaluronic acid (a), of the elastomer matrix based on poly(caprolactone-urea-urethane) alone (b), of the porous biomaterial according to the invention comprising the hyaluronic acid (c), and of the subtraction (d) of the c and b spectra.

FIG. 3 represents the mass loss and the mass absorption rate of the elastomer matrix based on poly(caprolactone-urea-urethane) alone (a, c) and of the porous biomaterial according to the invention comprising hyaluronic acid (b and d) upon in vitro degradation at 37° C. and accelerated at 55° C. and 75° C.

FIG. 4 represents the migration of cells (gingival fibroblast) from day 10 to day 40 within the elastomer matrix based on poly(caprolactone-urea-urethane) alone (elastomère) and the porous biomaterial according to the invention comprising hyaluronic acid (elastomère-AH).

FIG. 5 represents colonization by the cells (gingival fibroblast) after 20 days of migration within the poly(caprolactone-urea-urethane)-based elastomer matrix alone (elastomère) and of the porous biomaterial according to the invention comprising hyaluronic acid (elastomère-AH). (3D digital microscopy-hemalun staining).

FIG. 6 shows the appearance, after 10 days of culture, of the cells (gingival fibroblast) on the well bottom and at the periphery of the poly(caprolactone-urea-urethane)-based elastomer matrix alone (elastomère) and of the porous biomaterial according to the invention comprising hyaluronic acid (elastomère-AH). (Optical microscopy—×40 magnification).

FIG. 7 represents the cellularization, after 36 days of subcutaneous implantation in the rat, of the elastomer matrix based on poly(caprolactone-urea-urethane) alone (elastomère) and of the porous biomaterial according to the invention comprising hyaluronic acid (elastomère-AH). (Material marked by the white frame; †Neovascularization; *Multinucleated giant cells) (3D digital microscopy-hemalun/eosin staining).

FIG. 8 represents the structure of the collagens, after 36 days of subcutaneous implantation in the rat, within the elastomer matrix based on poly(caprolactone-urea-urethane) alone (elastomère) and on the porous biomaterial according to the invention comprising hyaluronic acid (elastomère-AH). (Material marked by the white box) (3D digital microscopy—picrosirius red staining—×4 and ×40 magnification).

FIG. 9 represents the labelling of the T-lymphocytes present, after 36 days of subcutaneous implantation in the rat, within the elastomer matrix based on poly(caprolactone-urea-urethane) alone (elastomère) and on the porous biomaterial according to the invention comprising hyaluronic acid (elastomère-AH). (Material marked by the black box) (3D digital microscopy—CD3 marking—×4 and ×40 magnification).

FIG. 10 represents the labelling of the macrophages present, after 36 days of subcutaneous implantation in the rat, within the elastomer matrix based on poly(caprolactone-urea-urethane) alone (elastomère) and the porous biomaterial according to the invention comprising hyaluronic acid (elastomère-AH). (Material marked by the black box) (3D digital microscopy—CD163 marking—×4 and ×40 magnification).

FIG. 11 represents the average values of optical density obtained after the hyaluronic acid has been stained with Alcian Blue for the elastomer matrix based on poly(caprolactone-urea-urethane) alone (elastomère) and on the porous biomaterial according to the invention comprising hyaluronic acid (elastomère-AH).

FIG. 12 represents the elastomer matrix based on poly(caprolactone-urea-urethane) alone (Elastomère) and on the porous biomaterial according to the invention comprising hyaluronic acid (Elastomère-AH) before and after beta radiation at 15 kGy. Images were obtained by 3D microscopy (VHX Keyence).

### EXAMPLES

### Example 1

### Formulation and Synthesis of the Porous Biomaterial According to the Invention

Initially, the high molecular weight hyaluronic acid was dissolved for 24 h at 37° C. in sterilized distilled water. The solution was then filtered through a 0.2 μm filter. In a second time, this aqueous solution was poured into the organic phase comprising the compounds necessary for the synthesis of the elastomer matrix based on poly(caprolactone-urea-urethane) in order to obtain a high internal phase emulsion. Subsequently, the polymerization/crosslinking of this emulsion leads to the production of the porous biomaterial according to the invention. Several concentrations of hyaluronic acid were tested. Several volume ratios of aqueous phase/organic phase were tested. Different synthesis temperatures have also been studied. These various parameters influence the size of the pores of the material. In the case of a use as gum substitutes, the retained scaffolds are those having pores having diameters ranging from 50 μm and 1400 μm with an average size of 600+/−170 μm. These materials are characterized in the examples below.

The formulation and the synthesis selected for obtaining the porous biomaterial according to the invention are:
Hyaluronic acid concentration in the aqueous phase: 1 mg/mL;
Aqueous phase/organic phase volume ratio: 92.5/7.5%,
Synthesis temperature: 18 h at 37° C. ; 4 h at 55° C. ; 2 h at 100° C.

### Example 2

### Physicochemical and Mechanical Properties of the Porous Biomaterial According to the Invention

The physicochemical properties of the porous biomaterials according to the invention obtained were tested by:
Fourier transform infrared (FTIR) spectroscopy for analyzing chemical functions present in synthesized biomaterials,
3D microscopy (VHX Keyence) for morphological observation of biomaterials;
Measurement of the volumetric absorption rates (rv) to determine the interconnectivity of the porous structure;
Measurement of the average molar mass between crosslinking nodes (Mc) by swelling and making it possible to evaluate the Young's modulus of the porous biomaterial (E₁*).

### 1. Interconnectivity/porosity

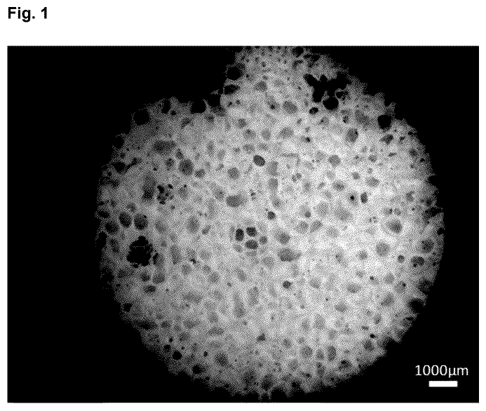

The images obtained by 3D microscopy (FIG. 1) show that the biomaterial according to the invention has a highly interconnected porous morphology (porosity=90+/−2%) (rv=100%) with multiscale pore sizes having diameters ranging from 50 μm and 1400 μm with an average size of 600+/−170 μm.

### 2. Chemical Composition and Hydrophilicity

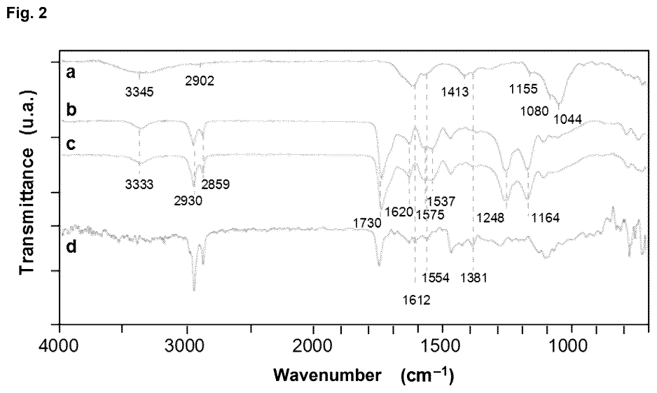

The FTIR analyses (FIG. 2) confirm the presence of hyaluronic acid in the poly(caprolactone-urea-urethane)-based elastomer matrix. The spectrum of the elastomer matrix based on poly(caprolactone-urea-urethane) alone exhibits the significant bands of these materials, such as the —NH groups of urethane at 3333 cm⁻¹, 1537 cm⁻¹ and 1248 cm⁻¹, the —C═O groups of urethane and of ester at 1330 cm⁻¹ and of urea at 1620 cm⁻¹, the —CNH group of urea at 1575 cm⁻¹, and the —COO ester groups at 1164 cm⁻¹.The subtraction of the spectra corresponding to the poly(caprolactone-urea-urethane)-based elastomer matrix alone and to the porous biomaterial according to the invention shows in the latter the presence of hyaluronic acid via in particular the presence of the 1612 cm⁻¹ typical band of the —CCH, —OCH and —COH groups of the polysaccharide cycle, as well as the 1554 cm⁻¹ and 1381 cm⁻¹ bands.

The incorporation of hyaluronic acid increases the hydrophilicity of the materials as attested to water contact angle measurements: θ=112+/−16° for the elastomeric matrix based on poly(caprolactone-urea-urethane) alone vs. θ=69+/−12° for the porous biomaterial comprising hyaluronic acid. The porous biomaterial according to the invention thus has a surface hydrophilicity more suitable for adhesion of fibroblasts, the latter having a greater adhesion to surfaces having a contact angle with water of between 60° and 80°.

Furthermore, the water absorption rate, obtained by immersing the materials in distilled water for 15 days, changes from about 400% for the elastomer matrix based on poly(caprolactone-urea-urethane) alone, to about 700% for the porous biomaterial comprising hyaluronic acid. This shows that the porous biomaterial according to the invention will be more suitable for liquid penetration and therefore cell infiltration.

### 3. Mechanical Properties

The average molecular weight between crosslinking nodes (Mc) was determined by toluene swell measurements. The elastomer matrix based on poly(caprolactone-urea-urethane) alone has a Mc value of 4860+/−240 g/mol, which makes it possible to evaluate the Young's modulus E₁* of the porous material. The value of 220+/−25 kPa attests to the elastomeric nature of the material. The porous biomaterial according to the invention comprising hyaluronic acid (of porosity and pore size equivalent to the poly(caprolactone-urea-urethane) based elastomer matrix alone) has a Mc value of 5380+/−1460 g/mol, which makes it possible to estimate the E₁* value at 123+/−11 kPa. Thus, hyaluronic acid participates in a slight decrease in the modulus of the porous biomaterial according to the invention, while retaining the elastomeric nature of the polymer matrix, which will allow the biomaterial according to the invention to resist the contraction forces exerted by the fibroblasts during cell migration within the material.

### 4. Degradation Kinetics

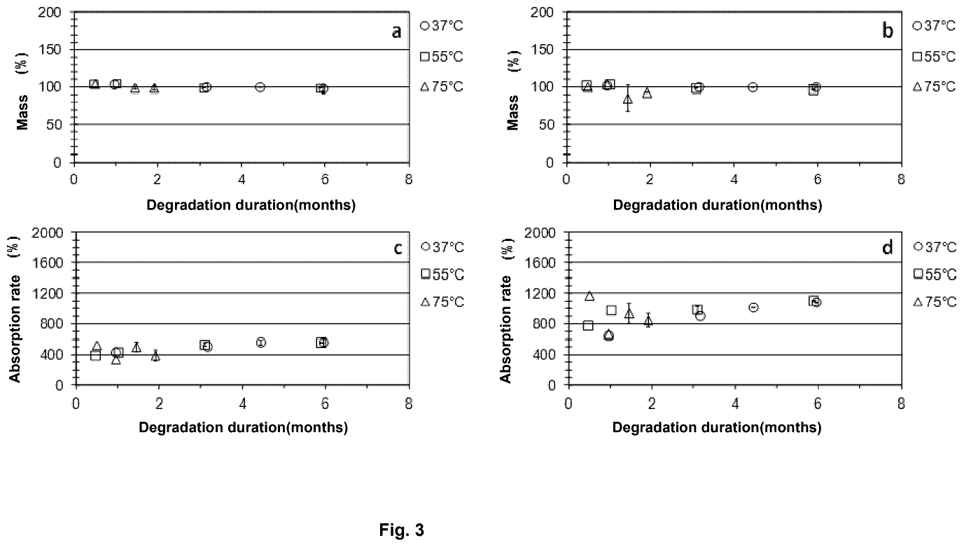

An important criterion during the production of a porous biomaterial for tissue engineering is its resorbability since it must be replaced over time by the newly formed tissue. In vitro degradation studies were carried out according to standard ISO 10993-13. The degradation kinetics were among others evaluated by measuring the loss of mass and the water adsorption rate at 37° C., 55° C. and 75° C. (FIG. 3). No difference was observed at 37° C. and 55° C. up to 6 months between the elastomer matrix based on poly(caprolactone-urea-urethane) alone and the biomaterial according to the invention comprising hyaluronic acid. The accelerated degradation tests at 75° C. have shown that the biomaterial according to the invention comprising hyaluronic acid degrades very slightly more rapidly than the elastomer matrix based on poly(caprolactone-urea-urethane) alone. This is due to an increase in the materials hydrophilicity. The biomaterial according to the invention is stable for more than 6 months at 37° C. The lifetime of a biomaterial is considerably reduced in vivo due to more stringent conditions; however, the biomaterial according to the invention is sufficiently stable for use in tissue engineering applications.

### Example 3

### Interactions Between the Porous Biomaterial According to the Invention and the Cells (Gingival Fibroblast)—In Vitro Study

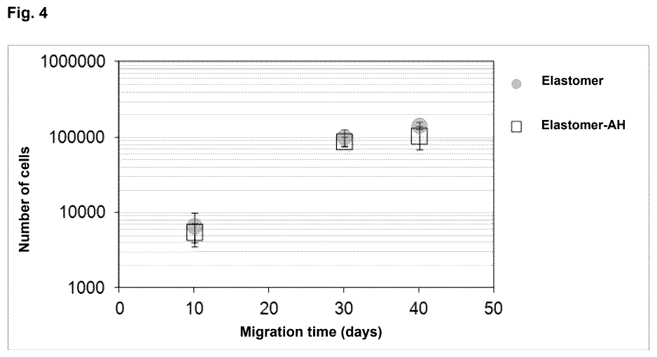

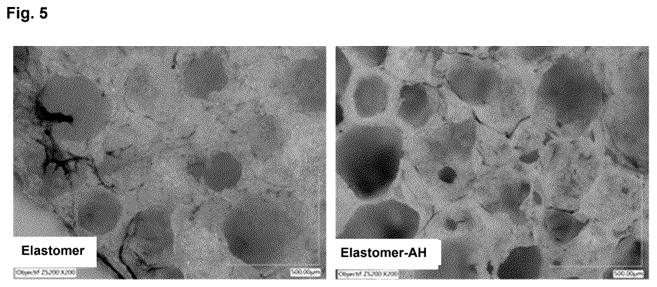

Colonization tests with gingival fibroblasts were carried out in order to test the “attracting” power of the porous biomaterial according to the invention comprising hyaluronic acid and of the elastomer matrix based on poly(caprolactone-urea-urethane) alone. The materials are deposited on a bed of gingival fibroblasts at 80% confluence. Migration of the cells was determined at 10, 30 and 40 days after contacting the materials. The cells present on and inside the materials are counted after separation of the cells by enzymatic treatment. The results obtained show that the cells are capable of migrating into the materials (FIG. 4). Gingival fibroblasts are able of migrating, proliferating and spreading at the surface of the pores of the materials (FIG. 5).

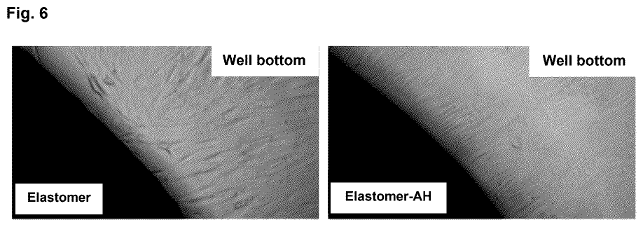

Interestingly, the cells present at the bottom of the well direct perpendicular to the material when they are at the periphery of the porous biomaterial according to the invention comprising hyaluronic acid (FIG. 6). Skin fibroblasts have been shown to spread and tend to align in the vicinity of skin fillers based on crosslinked hyaluronic acid, generally leading to an improvement in the fibroblast function (Quan et al., Journal of Investigative Dermatology, 2013, vol 133, pages 658-667). Although this result was rather attributed to structural reinforcement of the cutaneous extracellular matrix by the filling product, it is clear in view of our results that the porous biomaterial according to the invention comprising hyaluronic acid has an impact on the cells surrounding it.

### Example 4

### In Vivo Study of the Potential of the Porous Biomaterial According to the Invention for Soft Tissue Regeneration in a Subcutaneous Pouch Model in Rat

In vivo assays were performed by subcutaneous implantation of scaffolds along the dorsal midline of rats (Sprague-Dawley, 8-week male). The study makes it possible to evaluate the biocompatibility, the biointegration and the effectiveness of the porous biomaterial according to the invention during implantation.

In order to evaluate the effectiveness of the porous biomaterial according to the invention comprising hyaluronic acid with respect to the poly(caprolactone-urea-urethane)-based elastomer matrix alone, several batches of animals were monitored up to 36 days after implantation of said matrices subcutaneously. The effectiveness of the material according to the invention was evaluated by histological study of the materials taken after sacrifice of the animals. For each study time, 7 and 36 days, batches of 5 rats were made up (20 rats): group of elastomer matrix animals based on poly(caprolactone-urea-urethane) alone-group of elastomer matrix animals based on poly(caprolactone-urea-urethane) comprising the non-sulfated polysaccharide.

The subcutaneous pocket model consists in making a median incision in the back of the rat and in creating a subcutaneous pocket in which the material to be evaluated will be inserted.

### 1. Surgical Procedure

The animals are anaesthetized by intramuscular injection of 1.2 mL/kg of ketamine/xylazine (50/15 mg/kg). The dorsal part of the animals is toned and then disinfected with Betadine®. A median incision is made in the back of the rats and the skin flaps are raised bilaterally. The polymer matrices (1 cm in diameter over 2 to 3 mm in thickness) are inserted on either side of the median line and stabilized. The skin planes are then sutured with resorbable 5.0 sutures.

Animals are monitored daily, their general condition and behaviour being observed. Throughout the experiment, the animals showed no mobility loss, did not show signs of aggressiveness. Weight curves have evolved regularly. In the wound, there was no evidence of inflammation or necrosis.

### 2. Preparation of Implants

The poly(caprolactone-urea-urethane)-based elastomer matrices are removed from their storage medium (70% ethanol), rinsed with stirring for 5 minutes with physiological saline. They are then placed in the subcutaneous pocket.

### 3. Histology

After 7 and 36 days the animals are sacrificed. The elastomer matrices are removed, fixed with 10% paraformaldehyde, dried in increasing alcohol baths and then embedded in paraffin. 5 μM sections are then made with a manual microtome.

After dewaxing and rehydration, the sections are stained with heparan (hemalum: 0.2% hematein in a 5% aqueous solution of potassium alum/2% aqueous eosin) or with picrosirius red (0.1% picrosirius red in a saturated picric acid solution) for collagen detection.

### 4. Results

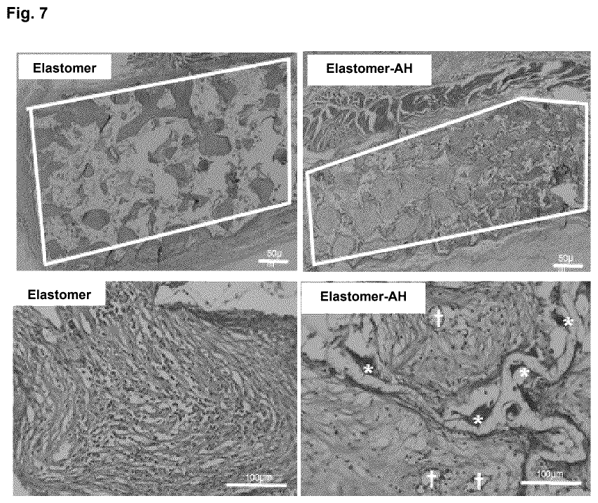

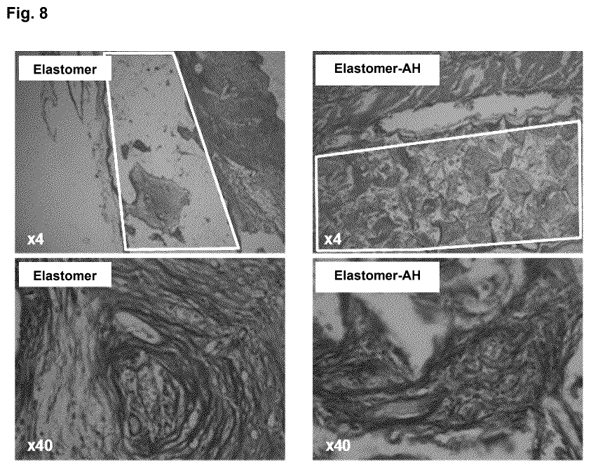

The pores of the biomaterials when implanted in the subcutaneous dorsal region in the laboratory rat are invaded by a fibrillar connective tissue as shown in the histological sections after staining with hemalum-eosin (FIG. 7) and with picrosirius red (FIG. 8).

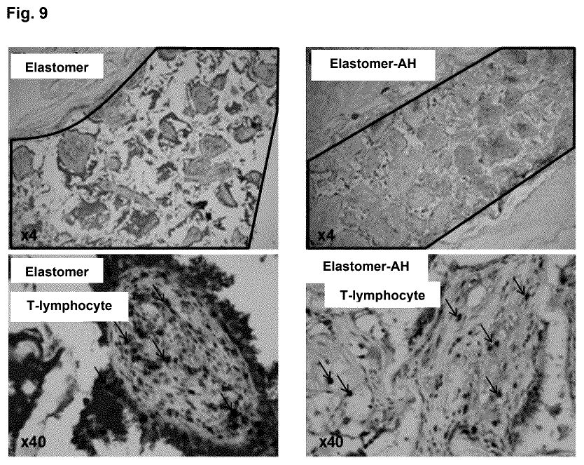

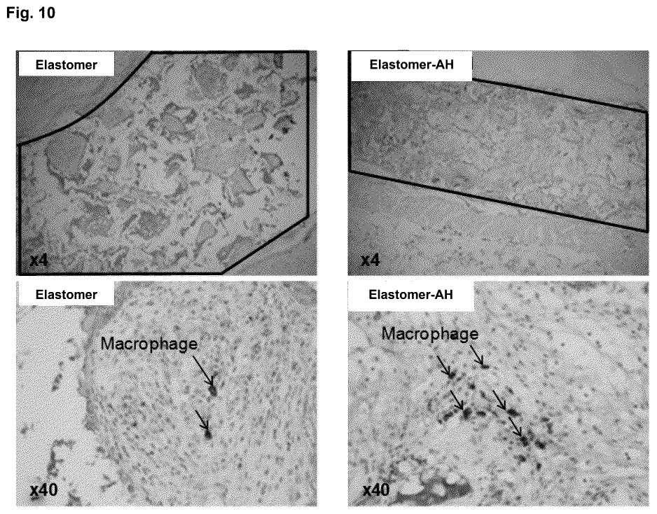

After implantation for 7 days, the third of the pores of the biomaterials closest to the surface are invaded by a fibrous connective tissue, numerous fibroblast-type cells are present therein. At 36 days post implantation, a little less than 50% of the pores are colonized in the elastomer matrix based on poly(caprolactone-urea-urethane) alone while approximately 100% is colonized in the porous biomaterial according to the invention comprising hyaluronic acid (FIG. 7). At high magnification, the pores of the elastomer matrix based on poly(caprolactone-urea-urethane) alone are invaded with a conjunctiva matrix which does not seem to be completely adherent to the surface of the pores. This surface appears colonized by many round nucleus cells which may be inflammatory cells as well as erythrocytes. In contrast, the pores of the porous biomaterial according to the invention comprising hyaluronic acid are invaded by a fibrous connective tissue remaining in contact with the surface of the pores. The number of round-core cells appears to be greatly decreased compared to the elastomer matrix alone indicating a decrease in the inflammatory component. Perfectly constituted vessels are present in the connective tissues, the red blood cells are well restricted thereto without signs of effusion. Multinucleated giant cells are also present at the surface of the pores and on the material itself. The connective tissue within the pores remains in contact with the biomaterial. At 36 days post-implantation, a decrease in the number of lymphocytes and macrophages is noted, more significantly in the porous biomaterial according to the invention comprising hyaluronic acid (FIG. 9 and FIG. 10). The porous biomaterial according to the invention comprising hyaluronic acid seems more compatible.

### Example 5

### Hyaluronic Acid Quantification Assay

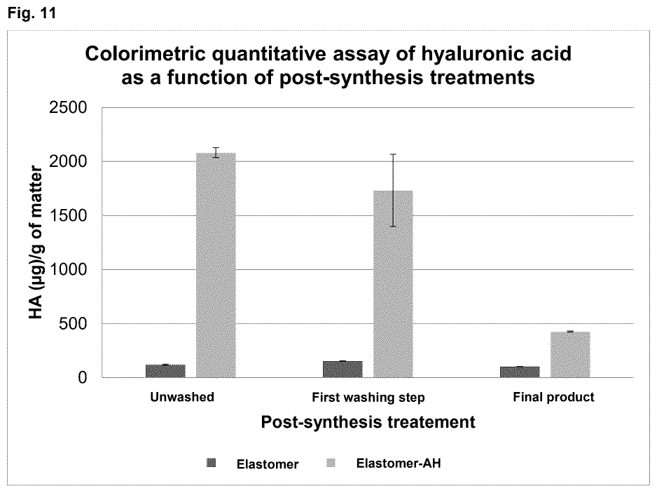

The hyaluronic acid quantification assay is performed by a colorimetric assay technique using Alcin Blue. Briefly, the elastomer matrices based on poly(caprolactone-urea-urethane) alone (Elastomère) and on the porous biomaterial according to the invention comprising hyaluronic acid (Elastomère-AH) are cut, weighed and then incubated 2 h in an Alcin Blue solution. The excess dye is removed and then substituted with a sodium acetate buffer solution (50 mM/MgCl250 mM at pH 5.8). The materials are then incubated in a 60% ethanol solution, then in an 80% acetic acid solution. Optical density is measured at 675 nm. Hyaluronic acid quantification assays were carried out at various steps of the manufacturing process and made it possible to determine an average concentration of 425 μg of HA/g of porous biomaterial according to the invention (see FIG. 11).

### Example 6

### Beta and Gamma Radiation Sterilization

Sterilisation by beta treatment was carried out by an ionization method consisting in continuously conveying, at a controlled speed, the biomaterial to the beta radiations emitted by an electron accelerator. The doses of 15, 25 and 45 Gy were tested. For example, the dose carried out at 25 kGy +/−10% was obtained under the following treatment conditions: frequency of 640 Hz/scanning setpoint of 2.6/number of revolutions: 1/speed: 0.898 m/min.

The sterilization by gamma treatment has been carried out by an ionization method consisting in exposing the biomaterial to gamma radiation emitted by a cobalt 60 source for a limited duration.

The dose performed was 25kGy+/−10 %.

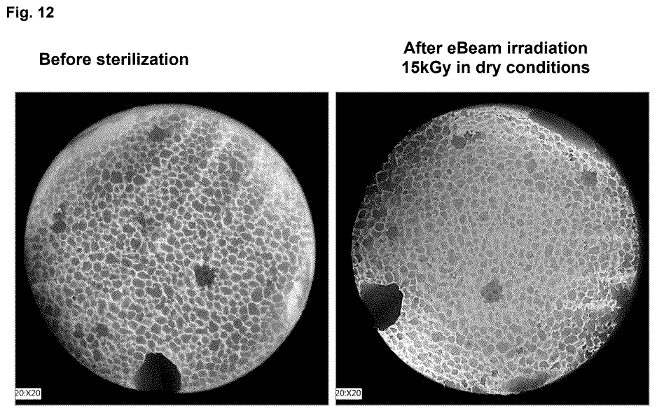

The images obtained by 3D microscopy (FIG. 12) show, for sterilization by beta radiation at 15 kGy that the elastomer matrix based on poly(caprolactone-urea-urethane) alone (Elastomère) and on the porous biomaterial according to the invention comprising hyaluronic acid (Elastomère-AH) does not show any structural alteration. The same results were obtained for the doses of beta and gamma radiations, of 15 to 45 kGy, whether the biomaterial according to the invention is dry or in an aqueous medium.

1. A porous biomaterial for tissue repair comprising:
at least one elastomer matrix, and
a non-sulfated polysaccharide,
wherein the isocyanate index of the elastomeric matrix is higher than 1.1.

2. The porous biomaterial according to claim 1, characterized in that the at least one elastomer matrix comprises an elastomer based on poly(ester-urea-urethane), the ester being chosen from caprolactone oligomers (PCL), lactic acid oligomers (PLA), glycolic acid oligomers (PGA), hydroxybutyrate oligomers (PHB), hydroxyvalerate oligomers (PVB), dioxanone oligomers (PDO), poly(ethylene adipate) oligomers (PEA), poly(butylene adipate) oligomers (PBA) or combinations thereof.

3. The porous biomaterial according to claim 1, characterized in that the non-sulfated polysaccharide is hyaluronic acid.

4. The porous biomaterial according to claim 1, characterized in that the elastomer matrix has an isocyanate index of between 1.1 and 6.0.

5. The porous biomaterial according to claim 1, characterized in that the isocyanate index of the elastomeric matric is between 1.2 and 4.5.

6. The porous biomaterial according to claim 1, characterized in that said biomaterial has a multiscale pore size of between 500 μm and 2000 μm.

7. The porous biomaterial according to claim 1, characterized in that said biomaterial has a total porosity greater than or equal to 60%.

8. The porous biomaterial according to claim 1, characterized in that said biomaterial has an interconnectivity between the pores of between 60% and 100%.

9. The porous biomaterial according to claim 1, characterized in that the amount of the non-sulphated polysaccharide represents between 0.05% and 2.0% by weight (w/w) of the porous biomaterial.

10. The porous biomaterial according to claim 1, characterized in that it comprises:
at least one elastomer matrix comprising an elastomer based on poly(caprolactone-urea-urethane), and
a non-sulfated polysaccharide,
characterized in that the non-sulfated polysaccharide is a hyaluronic acid having a molecular weight greater than or equal to 1000 kDa.

11. The porous biomaterial according to claim 1, characterized in that said biomaterial is in the form of a sponge, a film, a dressing, granules, monoliths or a membrane.

12. The porous biomaterial according to claim 1, for use for reinforcing, reconstructing and/or filling in tissue defects, advantageously for reinforcing, reconstructing and/or filling in soft tissue defects and/or epithelial tissues, advantageously for reinforcing, reconstructing and/or filling skin and/or mucous membrane defects.

13. The porous biomaterial according to claim 12, in which the reinforcement, the reconstruction and/or the tissue filling in is greater than or equal to 5% by volume of the volume of the tissue defect to be reinforced, reconstructed and/or filled in.

14. The porous biomaterial according to claim 1, for use in the reconstruction and/or reinforcement of gingival tissues.

15. The porous biomaterial according to claim 1, for use in the reconstruction and/or reinforcement of the visceral and/or pelvic and/or parietal tissues, advantageously in the treatment of pelvic organs prolapse, in the repair of pelvic tissues, in the reconstruction and/or reinforcement of the wall, in the reconstruction and/or reinforcement of a digestive wound.

16. The porous biomaterial according to claim 1, for its use in the treatment of burns, advantageously thermal burns, cold burns, electric burns, chemical burns, radiological burns and photochemical burns.

17. Process for preparing a biomaterial according to claim 14, in which the amount of non-sulfated polysaccharide represents between 0.05% and 2.0% (w/m) relative to the mass of aqueous liquid phase present in the emulsion.
